SEQUENCE LISTING (115 sequences)

SEQ ID NO: 1 - protein, 117 aa, synthetic construct
  source 1..117: note = h_Nb1 VHH
DVQLQESGGGLVQPGGSLRLSCAASGFTFSSYAMSWVRQAPGKGLEWVSDINSGGDNTDYADSVKGRFTISRDNAKNTLYLQMNSLKPEDTAVYYCARGPKLCFSSWGQGTQVTVSS

SEQ ID NO: 2 - protein, 8 aa, synthetic construct
  source 1..8: note = h_Nb1CDR1
GFTFSSYA

SEQ ID NO: 3 - protein, 8 aa, synthetic construct
  source 1..8: note = h_Nb1 CDR2
INSGGDNT

SEQ ID NO: 4 - protein, 10 aa, synthetic construct
  source 1..10: note = h_Nb1 CDR3
ARGPKLCFSS

SEQ ID NO: 5 - protein, 115 aa, synthetic construct
  source 1..115: note = h_Nb2 VHH
DVQLQESGGGLVQPGGSLRLSCAASGFTFGDRWMYWLRQAPGKGLEWVSSINPAGRSTYYADSVKGRFTISRDNAKNTLYLQMNSLKSEDTAVYYCTKDPGGYQKGQGTQVTVSS

SEQ ID NO: 6 - protein, 8 aa, synthetic construct
  source 1..8: note = h_Nb2 CDR1
GFTFGDRW

SEQ ID NO: 7 - protein, 8 aa, synthetic construct
  source 1..8: note = h_Nb2 CDR2
INPAGRST

SEQ ID NO: 8 - protein, 8 aa, synthetic construct
  source 1..8: note = h_Nb2 CDR3
TKDPGGYQ

SEQ ID NO: 9 - protein, 118 aa, synthetic construct
  source 1..118: note = h_Nb4 VHH
DVQLQESGGGLVQAGGSLRLSCAAPSRMFSGYTMGWYRQAPGKQRELVAQIANGGFTDYIDSVKGRFSISRDTVLNMMYLQMNSLKPEDTAVYYCNLVLVNGIPTRNWGQGTQVTVSS

SEQ ID NO: 10 - protein, 8 aa, synthetic construct
  source 1..8: note = h_Nb4 CDR1
SRMFSGYT

SEQ ID NO: 11 - protein, 7 aa, synthetic construct
  source 1..7: note = h_Nb4 CDR2
IANGGFT

SEQ ID NO: 12 - protein, 12 aa, synthetic construct
  source 1..12: note = h_Nb4 CDR3
NLVLVNGIPTRN

SEQ ID NO: 13 - protein, 127 aa, synthetic construct
  source 1..127: note = h_Nb5 VHH
DVQLQESGGGAVQSGESLRLSCAASGRDLMFYDVAWFRQAPGKEREGVSYIRSSGTLTKYADSVKGRFTISRDNDKNTVSLQMNSLNPEDTAKYYCAARLQSATSHSDLREDEFNSWGQGTQVTVSS

SEQ ID NO: 14 - protein, 8 aa, synthetic construct
  source 1..8: note = h_Nb5 CDR1
GRDLMFYD

SEQ ID NO: 15 - protein, 8 aa, synthetic construct
  source 1..8: note = h_Nb5 CDR2
IRSSGTLT

SEQ ID NO: 16 - protein, 20 aa, synthetic construct
  source 1..20: note = h_Nb5 CDR3
AARLQSATSHSDLREDEFNS

SEQ ID NO: 17 - protein, 120 aa, synthetic construct
  source 1..120: note = h_Nb6 VHH
DVQLQESGGGLAQPGGSLRLSCAASGFTFSSYAMSWARQAPGKGLEWVAGIYRGGSDTYYADSVKGRFTISRDNTKNTLYLQMNSLKPEDTALYYCIQGWGLGYANYDYWGQGTQVTVSS

SEQ ID NO: 18 - protein, 8 aa, synthetic construct
  source 1..8: note = h_Nb6 CDR2
IYRGGSDT

SEQ ID NO: 19 - protein, 13 aa, synthetic construct
  source 1..13: note = h_Nb6 CDR3
IQGWGLGYANYDY

SEQ ID NO: 20 - protein, 116 aa, synthetic construct
  source 1..116: note = h_Nb9 VHH
DVQLQESGGGLVQPGGSLRLSCASSGSIFGLGVKGWYRQPPGNQREMVAQIISGGRISYADSVKGRFTISRDNAKNTVYLQMNSLKPEDTAVYYCNAWGPGQRNYWGRGTQVTVSS

SEQ ID NO: 21 - protein, 8 aa, synthetic construct
  source 1..8: note = h_Nb9 CDR1
GSIFGLGV

SEQ ID NO: 22 - protein, 7 aa, synthetic construct
  source 1..7: note = h_Nb9 CDR2
IISGGRI

SEQ ID NO: 23 - protein, 10 aa, synthetic construct
  source 1..10: note = h_Nb9 CDR3
NAWGPGQRNY

SEQ ID NO: 24 - protein, 127 aa, synthetic construct
  source 1..127: note = h_Nb12 VHH
DVQLQESGGGLVQAGGSLRLSCAASGRTFSSRAMGWFRQAPGKEREFVAAISGSGRNTYYADSVKGRFTISRDNAKNSVNLQMNSLKPEDTAVYYCNAAGGGITIATMTQRTQYDYWGQGTQVTVSS

SEQ ID NO: 25 - protein, 8 aa, synthetic construct
  source 1..8: note = h_Nb12 CDR1
GRTFSSRA

SEQ ID NO: 26 - protein, 8 aa, synthetic construct
  source 1..8: note = h_Nb12 CDR2
ISGSGRNT

SEQ ID NO: 27 - protein, 20 aa, synthetic construct
  source 1..20: note = h_Nb12 CDR3
NAAGGGITIATMTQRTQYDY

SEQ ID NO: 28 - protein, 123 aa, synthetic construct
  source 1..123: note = h_Nb13 VHH
DVQLQESGGGLVQPGGSLRLSCAASGSIFSINAMGWYRQAPGKSRELVAAITSGGSTNYADSVKGRFTISRDTAKNTVYLLMNSLEPEDTAVYYCNVERRRPPHYLSEYDFDWGQGTQVTVSS

SEQ ID NO: 29 - protein, 8 aa, synthetic construct
  source 1..8: note = h_Nb13 CDR1
GSIFSINA

SEQ ID NO: 30 - protein, 7 aa, synthetic construct
  source 1..7: note = h_Nb13 CDR2
ITSGGST

SEQ ID NO: 31 - protein, 17 aa, synthetic construct
  source 1..17: note = h_Nb13 CDR3
NVERRRPPHYLSEYDFD

SEQ ID NO: 32 - protein, 124 aa, synthetic construct
  source 1..124: note = h_Nb19 VHH
DVQLQESGGGLVQAGGSLRLSCAASGRTFSGYAMAWFRQAPGKEREFVAAISGSGGRTYYADSRFTISRDNAKNTLYLQMNSLKPEDTAGYYCAAAGEGITIATMTQRTQYDYWGQGTQVTVSS

SEQ ID NO: 33 - protein, 8 aa, synthetic construct
  source 1..8: note = h_Nb19 CDR1
GRTFSGYA

SEQ ID NO: 34 - protein, 8 aa, synthetic construct
  source 1..8: note = h_Nb19 CDR2
ISGSGGRT

SEQ ID NO: 35 - protein, 20 aa, synthetic construct
  source 1..20: note = h_Nb19 CDR3
AAAGEGITIATMTQRTQYDY

SEQ ID NO: 36 - protein, 123 aa, synthetic construct
  source 1..123: note = h_Nb26 VHH
DVQLQESGGGLVQPGGSLRLSCAAPGSIFRINAMGWYRQAPGKQRELVAAITSGGSTNYADSVQGRFTISRDNAKNTVYLQMNSLKPEDTAVYYCNVERRRPPHYYSDYDFDWGQGTQVTVSS

SEQ ID NO: 37 - protein, 8 aa, synthetic construct
  source 1..8: note = h_Nb26 CDR1
GSIFRINA

SEQ ID NO: 38 - protein, 17 aa, synthetic construct
  source 1..17: note = h_Nb26 CDR3
NVERRRPPHYYSDYDFD

SEQ ID NO: 39 - protein, 121 aa, synthetic construct
  source 1..121: note = h_Nb30 VHH
DVQLQESGGGLVQPGESLRVSCAASGFTFSNYYMTWVRQAPGKGLEWVSFLNPSSSERFYADSVKGRFTISRDNAKNTLDLLMHSLKPEDTALYYCARHLKYNWAPNYDYWGQGTQVTVSS

SEQ ID NO: 40 - protein, 8 aa, synthetic construct
  source 1..8: note = h_Nb30 CDR1
GFTFSNYY

SEQ ID NO: 41 - protein, 8 aa, synthetic construct
  source 1..8: note = h_Nb30 CDR2
LNPSSSER

SEQ ID NO: 42 - protein, 14 aa, synthetic construct
  source 1..14: note = h_Nb30 CDR3
ARHLKYNWAPNYDY

SEQ ID NO: 43 - protein, 117 aa, synthetic construct
  source 1..117: note = h_Nb1_1 VHH
QVQLVESGGGLVQPGGSLRLSCAASGFTFSSYAMSWVRQAPGKGLEWVSAINSGGDNTYYADSVKGRFTISRDNSKNTLYLQMNSLRAEDTAVYYCARGPKLCFSSWGQGTLVTVSS

SEQ ID NO: 44 - protein, 117 aa, synthetic construct
  source 1..117: note = h_Nb1_2 VHH
QVQLVESGGGLVQPGGSLRLSCAASGFTFSSYAMSWVRQAPGKGLEWVSDINSGGDNTDYADSVKGRFTISRDNAKNTLYLQMNSLRAEDTAVYYCARGPKLCFSSWGQGTQVTVSS

SEQ ID NO: 45 - protein, 117 aa, synthetic construct
  source 1..117: note = h_Nb1_3 VHH
QVQLVESGGGLVQPGGSLRLSCAASGFTFSSYAMSWVRQAPGKGLEWVSDINSGGDNTDYADSVKGRFTISRDNSKNTLYLQMNSLRAEDTAVYYCARGPKLCFSSWGQGTQVTVSS

SEQ ID NO: 46 - protein, 117 aa, synthetic construct
  source 1..117: note = h_Nb1_4 VHH
QVQLVESGGGLVQPGGSLRLSCAASGFTFSSYAMSWVRQAPGKGLEWVSDINSGGDNTDYADSVKGRFTISRDNAKNTLYLQMNSLKAEDTAVYYCARGPKLCFSSWGQGTQVTVSS

SEQ ID NO: 47 - protein, 117 aa, synthetic construct
  source 1..117: note = h_Nb1_5 VHH
QVQLVESGGGLVQPGGSLRLSCAASGFTFSSYAMSWVRQAPGKGLEWVSDINSGGDNTDYADSVKGRFTISRDNSKNTLYLQMNSLKAEDTAVYYCARGPKLCFSSWGQGTQVTVSS

SEQ ID NO: 48 - protein, 115 aa, synthetic construct
  source 1..115: note = h_Nb2_1 VHH
QVQLVESGGGLVQPGGSLRLSCAASGFTFGDRWMYWVRQAPGKGLEWVSAINPAGRSTYYADSVKGRFTISRDNSKNTLYLQMNSLRAEDTAVYYCTKDPGGYQWGQGTLVTVSS

SEQ ID NO: 49 - protein, 115 aa, synthetic construct
  source 1..115: note = h_Nb2_2 VHH
QVQLLESGGGLVQPGGSLRLSCAASGFTFGDRWMYWLRQAPGKGLEWVSSINPAGRSTYYADSVKGRFTISRDNSKNTLYLQMNSLRAEDTAVYYCTKDPGGYQKGQGTQVTVSS

SEQ ID NO: 50 - protein, 115 aa, synthetic construct
  source 1..115: note = h_Nb2_3 VHH
QVQLVESGGGLVQPGGSLRLSCAASGFTFGDRWMYWLRQAPGKGLEWVSSINPAGRSTYYADSVKGRFTISRDNAKNSLYLQMNSLRAEDTAVYYCTKDPGGYQKGQGTQVTVSS

SEQ ID NO: 51 - protein, 115 aa, synthetic construct
  source 1..115: note = h_Nb2_4 VHH
QVQLLESGGGLVQPGGSLRLSCAASGFTFGDRWMYWVRQAPGKGLEWVSSINPAGRSTYYADSVKGRFTISRDNSKNTLYLQMNSLRAEDTAVYYCTKDPGGYQKGQGTQVTVSS

SEQ ID NO: 52 - protein, 115 aa, synthetic construct
  source 1..115: note = h_Nb2_5 VHH
QVQLVESGGGLVQPGGSLRLSCAASGFTFGDRWMYWVRQAPGKGLEWVSSINPAGRSTYYADSVKGRFTISRDNAKNSLYLQMNSLRAEDTAVYYCTKDPGGYQKGQGTQVTVSS

SEQ ID NO: 53 - protein, 118 aa, synthetic construct
  source 1..118: note = h_Nb4_1 VHH
QVQLVESGGGLVQPGGSLRLSCAASSRMFSGYTMGWVRQAPGKGLEWVSAIANGGFTYYADSVKGRFTISRDNSKNTLYLQMNSLRAEDTAVYYCNLVLVNGIPTRNWGQGTLVTVSS

SEQ ID NO: 54 - protein, 118 aa, synthetic construct
  source 1..118: note = h_Nb4_2 VHH
QVQLLESGGGLVQPGGSLRLSCAASSRMFSGYTMGWYRQAPGKGLEWVAQIANGGFTDYIDSVKGRFTISRDNSKNTMYLQMNSLRAEDTAVYYCNLVLVNGIPTRNWGQGTQVTVSS

SEQ ID NO: 55 - protein, 127 aa, synthetic construct
  source 1..127: note = h_Nb5_1 VHH
QVQLVESGGGLVQPGGSLRLSCAASGRDLMFYDVAWVRQAPGKGLEWVSAIRSSGTLTYYADSVKGRFTISRDNSKNTLYLQMNSLRAEDTAVYYCAARLQSATSHSDLREDEFNSWGQGTLVTVSS

SEQ ID NO: 56 - protein, 127 aa, synthetic construct
  source 1..127: note = h_Nb5_2 VHH
QVQLQESGGGAVQSGESLRLSCAASGRDLMFYDVAWFRQAPGKEREGVSYIRSSGTLTKYADSVKGRFTISRDNDKNTVSLQMNSLNPEDTAKYYCAARLQSATSHSDLREDEFNSWGQGTQVTVSS

SEQ ID NO: 57 - protein, 127 aa, synthetic construct
  source 1..127: note = h_Nb5_3 VHH
QVQLVESGGGLVQPGGSLRLSCAASGRDLMFYDVAWVRQAPGKGLEWVSYIRSSGTLTKYADSVKGRFTISRDNSKNTVYLQMNSLRAEDTAVYYCAARLQSATSHSDLREDEFNSWGQGTQVTVSS

SEQ ID NO: 58 - protein, 120 aa, synthetic construct
  source 1..120: note = h_Nb6_1 VHH
QVQLVESGGGLVQPGGSLRLSCAASGFTFSSYAMSWVRQAPGKGLEWVSAIYRGGSDTYYADSVKGRFTISRDNSKNTLYLQMNSLRAEDTAVYYCIQGWGLGYANYDYWGQGTLVTVSS

SEQ ID NO: 59 - protein, 120 aa, synthetic construct
  source 1..120: note = h_Nb6_2 VHH
QVQLLESGGGLVQPGGSLRLSCAASGFTFSSYAMSWARQAPGKGLEWVAGIYRGGSDTYYADSVKGRFTISRDNSKNTLYLQMNSLRAEDTAVYYCIQGWGLGYANYDYWGQGTQVTVSS

SEQ ID NO: 60 - protein, 114 aa, synthetic construct
  source 1..114: note = h_Nb9_1 VHH
QVQLVESGGGLVQPGGSLRLSCAASGSIFGLGVKGWVRQAPGKGLEWVSAIISGGRIYYADSVKGRFTISRDNSKNTLYLQMNSLRAEDTAVYYCNAWGPGQRNYWGQGTLVTV

SEQ ID NO: 61 - protein, 116 aa, synthetic construct
  source 1..116: note = h_Nb9_2 VHH
QVQLVESGGGLVQPGGSLRLSCASSGSIFGLGVKGWVRQAPGKGLEWVAQIISGGRISYADSVKGRFTISRDNAKNTVYLQMNSLRAEDTAVYYCNAWGPGQRNYWGRGTQVTVSS

SEQ ID NO: 62 - protein, 127 aa, synthetic construct
  source 1..127: note = h_Nb12_1 VHH
QVQLVESGGGLVQPGGSLRLSCAASGRTFSSRAMGWVRQAPGKGLEWVSAISGSGRNTYYADSVKGRFTISRDNSKNTLYLQMNSLRAEDTAVYYCNAAGGGITIATMTQRTQYDYWGQGTLVTVSS

SEQ ID NO: 63 - protein, 127 aa, synthetic construct
  source 1..127: note = h_Nb12_2 VHH
QVQLVESGGGLVQPGGSLRLSCAASGRTFSSRAMGWFRQAPGKELEFVAAISGSGRNTYYADSVKGRFTISRDNAKNSVYLQMNSLRAEDTAVYYCNAAGGGITIATMTQRTQYDYWGQGTQVTVSS

SEQ ID NO: 64 - protein, 123 aa, synthetic construct
  source 1..123: note = h_Nb13_1 VHH
QVQLVESGGGLVQPGGSLRLSCAASGSIFSINAMGWVRQAPGKGLEWVSAITSGGSTYYADSVKGRFTISRDNSKNTLYLQMNSLRAEDTAVYYCNVERRRPPHYLSEYDFDWGQGTLVTVSS

SEQ ID NO: 65 - protein, 123 aa, synthetic construct
  source 1..123: note = h_Nb13_2 VHH
EVQLVESGGGLVQPGGSLRLSCAASGSIFSINAMGWVRQAPGKGLEWVAAITSGGSTNYADSVKGRFTISRDNAKNTVYLQMNSLRAEDTAVYYCNVERRRPPHYLSEYDFDWGQGTQVTVSS

SEQ ID NO: 66 - protein, 124 aa, synthetic construct
  source 1..124: note = h_Nb19_1 VHH
QVQLVESGGGLVQPGGSLRLSCAASGRTFSGYAMSWVRQAPGKGLEWVSAISGSGYYADSVKGRFTISRDNSKNTLYLQMNSLRAEDTAVYYCAAAGEGITIATMTQRTQYDYWGQGTLVTVSS

SEQ ID NO: 67 - protein, 124 aa, synthetic construct
  source 1..124: note = h_Nb19_2 VHH
QVQLVESGGGLVQPGGSLRLSCAASGRTFSGYAMAWFRQAPGKGREFVAAISGSGGRTYYADSRFTISRDNAKNTLYLQMNSLRAEDTAVYYCAAAGEGITIATMTQRTQYDYWGQGTQVTVSS

SEQ ID NO: 68 - protein, 123 aa, synthetic construct
  source 1..123: note = h_Nb26_1 VHH
QVQLVESGGGLVQPGGSLRLSCAASGSIFRINAMGWVRQAPGKGLEWVSAITSGGSTYYADSVKGRFTISRDNSKNTLYLQMNSLRAEDTAVYYCNVERRRPPHYYSDYDFDWGQGTLVTVSS

SEQ ID NO: 69 - protein, 123 aa, synthetic construct
  source 1..123: note = h_Nb26_2 VHH
QVQLVQSGAEVKKPGASVKVSCKASGSIFRINAMGWYRQAPGQGLEWVAAITSGGSTNYADSVQGRFTITRDNSTSTVYLELRSLRSDDTAVYYCNVERRRPPHYYSDYDFDWGQGTQVTVSS

SEQ ID NO: 70 - protein, 121 aa, synthetic construct
  source 1..121: note = h_Nb30_1 VHH
QVQLVESGGGLVQPGGSLRLSCAASGFTFSNYYMSWVRQAPGKGLEWVSALNPSSSERYYADSVKGRFTISRDNSKNTLYLQMNSLRAEDTAVYYCARHLKYNWAPNYDYWGQGTLVTVSS

SEQ ID NO: 71 - protein, 121 aa, synthetic construct
  source 1..121: note = h_Nb30_2 VHH
EVQLVESGGGLVQPGGSLRLSCAASGFTFSNYYMTWVRQAPGKGLEWVSFLNPSSSERFYADSVKGRFTISRDNAKNTLDLQMNSLRAEDTAVYYCARHLKYNWAPNYDYWGQGTQVTVSS

SEQ ID NO: 72 - protein, 117 aa, synthetic construct
  source 1..117: note = h_Nb1_A61N VHH
DVQLQESGGGLVQPGGSLRLSCAASGFTFSSYAMSWVRQAPGKGLEWVSDINSGGDNTDYNDSVKGRFTISRDNAKNTLYLQMNSLKPEDTAVYYCARGPKLCFSSWGQGTQVTVSS

SEQ ID NO: 73 - protein, 117 aa, synthetic construct
  source 1..117: note = h_Nb1_A61Q VHH
DVQLQESGGGLVQPGGSLRLSCAASGFTFSSYAMSWVRQAPGKGLEWVSDINSGGDNTDYQDSVKGRFTISRDNAKNTLYLQMNSLKPEDTAVYYCARGPKLCFSSWGQGTQVTVSS

SEQ ID NO: 74 - protein, 117 aa, synthetic construct
  source 1..117: note = h_Nb1_A61K VHH
DVQLQESGGGLVQPGGSLRLSCAASGFTFSSYAMSWVRQAPGKGLEWVSDINSGGDNTDYKDSVKGRFTISRDNAKNTLYLQMNSLKPEDTAVYYCARGPKLCFSSWGQGTQVTVSS

SEQ ID NO: 75 - protein, 117 aa, synthetic construct
  source 1..117: note = h_Nb1_D59R VHH
DVQLQESGGGLVQPGGSLRLSCAASGFTFSSYAMSWVRQAPGKGLEWVSDINSGGDNTRYADSVKGRFTISRDNAKNTLYLQMNSLKPEDTAVYYCARGPKLCFSSWGQGTQVTVSS

SEQ ID NO: 76 - protein, 117 aa, synthetic construct
  source 1..117: note = h_Nb1_D59M VHH
DVQLQESGGGLVQPGGSLRLSCAASGFTFSSYAMSWVRQAPGKGLEWVSDINSGGDNTMYADSVKGRFTISRDNAKNTLYLQMNSLKPEDTAVYYCARGPKLCFSSWGQGTQVTVSS

SEQ ID NO: 77 - protein, 117 aa, synthetic construct
  source 1..117: note = h_Nb1_D59F VHH
DVQLQESGGGLVQPGGSLRLSCAASGFTFSSYAMSWVRQAPGKGLEWVSDINSGGDNTFYADSVKGRFTISRDNAKNTLYLQMNSLKPEDTAVYYCARGPKLCFSSWGQGTQVTVSS

SEQ ID NO: 78 - protein, 117 aa, synthetic construct
  source 1..117: note = h_Nb1_D62W VHH
DVQLQESGGGLVQPGGSLRLSCAASGFTFSSYAMSWVRQAPGKGLEWVSDINSGGDNTDYAWSVKGRFTISRDNAKNTLYLQMNSLKPEDTAVYYCARGPKLCFSSWGQGTQVTVSS

SEQ ID NO: 79 - protein, 117 aa, synthetic construct
  source 1..117: note = h_Nb1_D62Y VHH
DVQLQESGGGLVQPGGSLRLSCAASGFTFSSYAMSWVRQAPGKGLEWVSDINSGGDNTDYAYSVKGRFTISRDNAKNTLYLQMNSLKPEDTAVYYCARGPKLCFSSWGQGTQVTVSS

SEQ ID NO: 80 - protein, 117 aa, synthetic construct
  source 1..117: note = h_Nb1_C103K VHH
DVQLQESGGGLVQPGGSLRLSCAASGFTFSSYAMSWVRQAPGKGLEWVSDINSGGDNTDYADSVKGRFTISRDNAKNTLYLQMNSLKPEDTAVYYCARGPKLKFSSWGQGTQVTVSS

SEQ ID NO: 81 - protein, 117 aa, synthetic construct
  source 1..117: note = h_Nb1_C103M VHH
DVQLQESGGGLVQPGGSLRLSCAASGFTFSSYAMSWVRQAPGKGLEWVSDINSGGDNTDYADSVKGRFTISRDNAKNTLYLQMNSLKPEDTAVYYCARGPKLMFSSWGQGTQVTVSS

SEQ ID NO: 82 - protein, 117 aa, synthetic construct
  source 1..117: note = h_Nb1_C103W VHH
DVQLQESGGGLVQPGGSLRLSCAASGFTFSSYAMSWVRQAPGKGLEWVSDINSGGDNTDYADSVKGRFTISRDNAKNTLYLQMNSLKPEDTAVYYCARGPKLWFSSWGQGTQVTVSS

SEQ ID NO: 83 - protein, 117 aa, synthetic construct
  source 1..117: note = h_Nb1_G99R VHH
DVQLQESGGGLVQPGGSLRLSCAASGFTFSSYAMSWVRQAPGKGLEWVSDINSGGDNTDYADSVKGRFTISRDNAKNTLYLQMNSLKPEDTAVYYCARRPKLCFSSWGQGTQVTVSS

SEQ ID NO: 84 - protein, 117 aa, synthetic construct
  source 1..117: note = h_Nb1_G99Q VHH
DVQLQESGGGLVQPGGSLRLSCAASGFTFSSYAMSWVRQAPGKGLEWVSDINSGGDNTDYADSVKGRFTISRDNAKNTLYLQMNSLKPEDTAVYYCARQPKLCFSSWGQGTQVTVSS

SEQ ID NO: 85 - protein, 117 aa, synthetic construct
  source 1..117: note = h_Nb1_G99W VHH
DVQLQESGGGLVQPGGSLRLSCAASGFTFSSYAMSWVRQAPGKGLEWVSDINSGGDNTDYADSVKGRFTISRDNAKNTLYLQMNSLKPEDTAVYYCARWPKLCFSSWGQGTQVTVSS

SEQ ID NO: 86 - protein, 117 aa, synthetic construct
  source 1..117: note = h_Nb1_G99Y VHH
DVQLQESGGGLVQPGGSLRLSCAASGFTFSSYAMSWVRQAPGKGLEWVSDINSGGDNTDYADSVKGRFTISRDNAKNTLYLQMNSLKPEDTAVYYCARYPKLCFSSWGQGTQVTVSS

SEQ ID NO: 87 - protein, 115 aa, synthetic construct
  source 1..115: note = h_Nb2_G30R VHH
DVQLQESGGGLVQPGGSLRLSCAASGFTFRDRWMYWLRQAPGKGLEWVSSINPAGRSTYYADSVKGRFTISRDNAKNTLYLQMNSLKSEDTAVYYCTKDPGGYQKGQGTQVTVSS

SEQ ID NO: 88 - protein, 115 aa, synthetic construct
  source 1..115: note = h_Nb2_G30F VHH
DVQLQESGGGLVQPGGSLRLSCAASGFTFFDRWMYWLRQAPGKGLEWVSSINPAGRSTYYADSVKGRFTISRDNAKNTLYLQMNSLKSEDTAVYYCTKDPGGYQKGQGTQVTVSS

SEQ ID NO: 89 - protein, 115 aa, synthetic construct
  source 1..115: note = h_Nb2_G30W VHH
DVQLQESGGGLVQPGGSLRLSCAASGFTFWDRWMYWLRQAPGKGLEWVSSINPAGRSTYYADSVKGRFTISRDNAKNTLYLQMNSLKSEDTAVYYCTKDPGGYQKGQGTQVTVSS

SEQ ID NO: 90 - protein, 115 aa, synthetic construct
  source 1..115: note = h_Nb2_G101R VHH
DVQLQESGGGLVQPGGSLRLSCAASGFTFGDRWMYWLRQAPGKGLEWVSSINPAGRSTYYADSVKGRFTISRDNAKNTLYLQMNSLKSEDTAVYYCTKDPRGYQKGQGTQVTVSS

SEQ ID NO: 91 - protein, 115 aa, synthetic construct
  source 1..115: note = h_Nb2_G101W VHH
DVQLQESGGGLVQPGGSLRLSCAASGFTFGDRWMYWLRQAPGKGLEWVSSINPAGRSTYYADSVKGRFTISRDNAKNTLYLQMNSLKSEDTAVYYCTKDPWGYQKGQGTQVTVSS

SEQ ID NO: 92 - protein, 115 aa, synthetic construct
  source 1..115: note = h_Nb2_G101Y VHH
DVQLQESGGGLVQPGGSLRLSCAASGFTFGDRWMYWLRQAPGKGLEWVSSINPAGRSTYYADSVKGRFTISRDNAKNTLYLQMNSLKSEDTAVYYCTKDPGYYQKGQGTQVTVSS

SEQ ID NO: 93 - protein, 115 aa, synthetic construct
  source 1..115: note = h_Nb2_F29R VHH
DVQLQESGGGLVQPGGSLRLSCAASGFTRGDRWMYWLRQAPGKGLEWVSSINPAGRSTYYADSVKGRFTISRDNAKNTLYLQMNSLKSEDTAVYYCTKDPGGYQKGQGTQVTVSS

SEQ ID NO: 94 - protein, 115 aa, synthetic construct
  source 1..115: note = h_Nb2_F29W VHH
DVQLQESGGGLVQPGGSLRLSCAASGFTWGDRWMYWLRQAPGKGLEWVSSINPAGRSTYYADSVKGRFTISRDNAKNTLYLQMNSLKSEDTAVYYCTKDPGGYQKGQGTQVTVSS

SEQ ID NO: 95 - protein, 115 aa, synthetic construct
  source 1..115: note = h_Nb2_P100R VHH
DVQLQESGGGLVQPGGSLRLSCAASGFTFGDRWMYWLRQAPGKGLEWVSSINPAGRSTYYADSVKGRFTISRDNAKNTLYLQMNSLKSEDTAVYYCTKDRGGYQKGQGTQVTVSS

SEQ ID NO: 96 - protein, 115 aa, synthetic construct
  source 1..115: note = h_Nb2_P100Q VHH
DVQLQESGGGLVQPGGSLRLSCAASGFTFGDRWMYWLRQAPGKGLEWVSSINPAGRSTYYADSVKGRFTISRDNAKNTLYLQMNSLKSEDTAVYYCTKDQGGYQKGQGTQVTVSS

SEQ ID NO: 97 - protein, 115 aa, synthetic construct
  source 1..115: note = h_Nb2_P100E VHH
DVQLQESGGGLVQPGGSLRLSCAASGFTFGDRWMYWLRQAPGKGLEWVSSINPAGRSTYYADSVKGRFTISRDNAKNTLYLQMNSLKSEDTAVYYCTKDEGGYQKGQGTQVTVSS

SEQ ID NO: 98 - protein, 10 aa, synthetic construct
  source 1..10: note = h_Nb1_C103K CDR3
ARGPKLKFSS

SEQ ID NO: 99 - protein, 10 aa, synthetic construct
  source 1..10: note = h_Nb1_C103M CDR3
ARGPKLMFSS

SEQ ID NO: 100 - protein, 10 aa, synthetic construct
  source 1..10: note = h_Nb1_C103W CDR3
ARGPKLWFSS

SEQ ID NO: 101 - protein, 10 aa, synthetic construct
  source 1..10: note = h_Nb1_G99R CDR3
ARRPKLCFSS

SEQ ID NO: 102 - protein, 10 aa, synthetic construct
  source 1..10: note = h_Nb1_G99Q CDR3
ARQPKLCFSS

SEQ ID NO: 103 - protein, 10 aa, synthetic construct
  source 1..10: note = h_Nb1_G99W CDR3
ARWPKLCFSS

SEQ ID NO: 104 - protein, 10 aa, synthetic construct
  source 1..10: note = h_Nb1_G99Y CDR3
ARYPKLCFSS

SEQ ID NO: 105 - protein, 8 aa, synthetic construct
  source 1..8: note = h_Nb2_G30R CDR1
GFTFRDRW

SEQ ID NO: 106 - protein, 8 aa, synthetic construct
  source 1..8: note = h_Nb2_G30F CDR1
GFTFFDRW

SEQ ID NO: 107 - protein, 8 aa, synthetic construct
  source 1..8: note = h_Nb2_G30W CDR1
GFTFWDRW

SEQ ID NO: 108 - protein, 8 aa, synthetic construct
  source 1..8: note = h_Nb2_G101R CDR3
TKDPRGYQ

SEQ ID NO: 109 - protein, 8 aa, synthetic construct
  source 1..8: note = h_Nb2_G101W CDR3
TKDPWGYQ

SEQ ID NO: 110 - protein, 8 aa, synthetic construct
  source 1..8: note = h_Nb2_G101Y CDR3
TKDPYGYQ

SEQ ID NO: 111 - protein, 8 aa, synthetic construct
  source 1..8: note = h_Nb2_F29R CDR1
GFTRGDRW

SEQ ID NO: 112 - protein, 8 aa, synthetic construct
  source 1..8: note = h_Nb2_F29W CDR1
GFTWGDRW

SEQ ID NO: 113 - protein, 8 aa, synthetic construct
  source 1..8: note = h_Nb2_P100R CDR3
TKDRGGYQ

SEQ ID NO: 114 - protein, 8 aa, synthetic construct
  source 1..8: note = h_Nb2_P100Q CDR3
TKDQGGYQ

SEQ ID NO: 115 - protein, 8 aa, synthetic construct
  source 1..8: note = h_Nb2_P100E CDR3
TKDEGGYQ

(19) United States
(12) Patent Application Publication
(10) Pub. No.: US 2025/0197503 A1
(43) Pub. Date: Jun. 19, 2025

(54) ANTI-HUMAN PD-Ll AND TLR7 DOUBLE-TARGETING NANOBODY CONJUGATE DRUG AND USE THEREOF IN RESISTING TUMOR

(71) Applicant: SHANGHAI INSTITUTE OF MATERIA MEDICA , CHINESE ACADEMY OF SCIENCES, Shanghai (CN)

(72) Inventors: Likun GONG, Shanghai (CN); Xiaolu YU, Shanghai (CN); Wei HUANG, Shanghai (CN); Jia LI, Shanghai (CN); Yiru LONG, Shanghai (CN); Feng TANG, Shanghai (CN); Chao HU, Shanghai (CN); Fanglin LI, Shanghai (CN); Zhi LIU, Shanghai (CN); Qiuping QIN, Shanghai (CN); Jianhua SUN, Shanghai (CN); Junjiu XU, Shanghai (CN)

(21) Appl. No.: 18/847,303

(22) PCT Filed: Mar. 14, 2023
(86) PCT No.: PCT/CN2023/081459

§ 371 Date: Sep. 16, 2024

(30) Foreign Application Priority Data
Mar. 15, 2022 (CN) 202210254773.7
Sep. 16, 2022 (CN) 202211130623.1

(51) Int. Cl. C07K 16/28; A61K 47/68; A61P 35/00

(52) U.S. Cl. CPC C07K 16/2827; A61K 47/6803; A61K 47/6849; A61P 35/00; C07K 2317/24; C07K 2317/569; C07K 2317/76

(57) ABSTRACT

An anti-human PD-L1 nanobody and use thereof, and an anti-human PD-L1 and TLR7 double-targeting nanobody conjugate drug, a preparation method therefor and use thereof are provided. Specifically, provided are use and a solution of an anti-human PD-L1 nanobody and a derivative protein thereof for anti-tumor treatment, and also provided are design, preparation, and identification solutions for a novel anti-human PD-L1 and TLR7 double-targeting nanobody drug conjugate and a derivative molecule thereof and an effect thereof in anti-tumor treatment. The anti-human PD-L1 and TLR7 double-targeting nanobody drug conjugate can exert significant anti-tumor efficacy.

### TECHNICAL FIELD

The present invention relates to the field of biomedicine, and more specifically to an anti-human PD-L1 and TLR7 dual-targeting nanobody conjugate drug and use thereof against a tumor.

### BACKGROUND

PD-1/PD-L1 immune checkpoint blockade therapy (ICB) is effective against many types of cancers, but it has problems such as low response rate and drug resistance and recurrence. Therefore, determining appropriate treatment strategies to improve efficacy is an important task in tumor immunotherapy.

Toll-like receptor (TLR) is an important target of innate immunity, which is mainly expressed on antigen presenting cells (APC) and natural killer cells (NK). TLR agonists can effectively activate APC, increase phagocytic function and antigen presentation function, thus promoting T cell activation and being effective in tumor suppression. In addition, using TLR agonists in tumor immunity is expected to turn cold tumors hot and solve the problem of low response rate of single immune checkpoint inhibitors.

Antibody miniaturization is one of the research directions in antibody genetic engineering. Nanobodies, namely heavy chain single domain antibodies VHH, are the variable regions of natural heavy chain antibodies lacking light chains from camelids (camels, llamas, alpacas and their close relatives), and are the smallest naturally occurring antigen-binding units known so far. Compared with monoclonal antibodies, nanobodies have superior tumor targeting and stability, and can achieve a targeted delivery of drugs and thus achieving precision therapy.

In summary, there is an urgent need to develop a novel and effective anti-human PD-L1 nanobody and TLR7 conjugate drug in this field.

### SUMMARY OF THE INVENTION

The object of the present invention is to provide a novel anti-human PD-L1 nanobody drug.

The object of the present invention is to provide a novel and effective anti-human PD-L1 nanobody conjugate drug.

The object of the present invention is to provide a combined use of an anti-human PD-L1 nanobody and TLR7 agonist for anti-tumor treatment and to provide a PD-L1 and TLR7 dual-targeting nanobody conjugate drug.

Another object of the present invention is to provide a use of an anti-human PD-L1 and TLR7 dual-targeting nanobody conjugate drug in the prevention and treatment of tumors, especially tumors with a low response rate to PD-L1 antibodies.

In the first aspect of the present invention, it provides an antibody-drug conjugate or a pharmaceutically acceptable salt thereof, wherein the structure of the antibody-drug conjugate is as shown in formula I:

Ab-(J-U)ₙ (I)
wherein,
Ab is a PD-L1 antibody;
each U is independently a TLR agonist;
J is a chemical bond or linker;
n is 0 or a positive integer; and
“—” is a chemical bond, a connector or a linker.

In another preferred embodiment, the PD-L1 antibody includes a monospecific antibody, a bispecific antibody, and a multispecific antibody (such as a trispecific antibody).

In another preferred embodiment, the PD-L1 antibody comprises: a monoclonal antibody, single chain antibody (scFv), and nanobody.

In another preferred embodiment, the PD-L1 antibody comprises a monovalent, bivalent or multivalent antibody.

In another preferred embodiment, the PD-L1 antibody comprises an antibody in a multimeric form.

In another preferred embodiment, the PD-L1 antibody specifically binds to PD-L1.

In another preferred embodiment, the PD-L1 antibody comprises a PD-L1 monovalent nanobody, bivalent nanobody and/or multivalent nanobody.

In another preferred embodiment, the PD-L1 antibody comprises a blocking type (which blocks the binding of PD-L1 and PD-1), a non-blocking type (which does not block the binding of PD-L1 and PD-1), or a combination thereof.

In another preferred embodiment, the PD-L1 antibody is a blocking type antibody.

In another preferred embodiment, the PD-L1 antibody blocks the binding of PD-1 to PD-L1.

In another preferred embodiment, the PD-L1 is a human PD-L1 or PD-L1 of a non-human mammal (such as mouse PD-L1).

In another preferred embodiment, the PD-L1 antibody is a human or non-human mammal antibody.

In another preferred embodiment, the non-human mammal is selected from the group consisting of camel, alpaca, mouse, and cynomolgus monkey.

In another preferred embodiment, the PD-L1 antibody is a PD-L1 nanobody or a derivative antibody thereof, preferably a nanobody targeting human PD-L1 or a derivative antibody thereof.

In another preferred embodiment, the derivative antibody is a modified PD-L1 nanobody, including but not limited to a bivalent antibody and/or a multivalent antibody formed by connecting the PD-L1 nanobody to an Fc fragment, human serum albumin, polyethylene glycol PEG.

In another preferred embodiment, the nanobody comprises a humanized antibody, camel-derived antibody, and chimeric antibody.

In another preferred embodiment, the PD-L1 nanobody is a nanobody that specifically binds to human PD-L1, and the complementary determining region CDR of the VHH chain in the nanobody is selected from the group consisting of:
(1) CDR1 shown in SEQ ID NO: 2, CDR2 shown in SEQ ID NO: 3, and CDR3 shown in SEQ ID NO: 4;
(2) CDR1 shown in SEQ ID NO: 6, CDR2 shown in SEQ ID NO: 7, and CDR3 shown in SEQ ID NO: 8;
(3) CDR1 shown in SEQ ID NO: 10, CDR2 shown in SEQ ID NO: 11, and CDR3 shown in SEQ ID NO: 12;
(4) CDR1 shown in SEQ ID NO: 14, CDR2 shown in SEQ ID NO: 15, and CDR3 shown in SEQ ID NO: 16;
(5) CDR1 shown in SEQ ID NO: 2, CDR2 shown in SEQ ID NO: 18, and CDR3 shown in SEQ ID NO: 19;
(6) CDR1 shown in SEQ ID NO: 21, CDR2 shown in SEQ ID NO: 22, and CDR3 shown in SEQ ID NO: 23;
(7) CDR1 shown in SEQ ID NO: 25, CDR2 shown in SEQ ID NO: 26, and CDR3 shown in SEQ ID NO: 27;
(8) CDR1 shown in SEQ ID NO: 29, CDR2 shown in SEQ ID NO: 30, and CDR3 shown in SEQ ID NO: 31;
(9) CDR1 shown in SEQ ID NO: 33, CDR2 shown in SEQ ID NO: 34, and CDR3 shown in SEQ ID NO: 35;
(10) CDR1 shown in SEQ ID NO: 37, CDR2 shown in SEQ ID NO: 30, and CDR3 shown in SEQ ID NO: 38;
(11) CDR1 shown in SEQ ID NO: 40, CDR2 shown in SEQ ID NO: 41, and CDR3 shown in SEQ ID NO: 42;

In another preferred embodiment, any one of the above amino acid sequences further comprises a derivative sequence that is optionally added, deleted, modified and/or substituted with at least one (such as 1-3, preferably 1-2, more preferably 1) amino acid and may retain the ability to bind to PD-L1.

In another preferred embodiment, the PD-L1 nanobody is a nanobody that specifically binds to human PD-L1, and the complementary determining region CDR of the VHH chain in the nanobody is selected from the group consisting of:
(1) CDR1 shown in SEQ ID NO: 2, CDR2 shown in SEQ ID NO: 3, and CDR3 shown in SEQ ID NO: 4;
(2) CDR1 shown in SEQ ID NO: 6, CDR2 shown in SEQ ID NO: 7, and CDR3 shown in SEQ ID NO: 8;
(3) CDR1 shown in SEQ ID NO: 2, CDR2 shown in SEQ ID NO: 3, and CDR3 shown in SEQ ID NO: 98;
(4) CDR1 shown in SEQ ID NO: 2, CDR2 shown in SEQ ID NO: 3, and CDR3 shown in SEQ ID NO: 99;
(5) CDR1 shown in SEQ ID NO: 2, CDR2 shown in SEQ ID NO: 3, and CDR3 shown in SEQ ID NO: 100;
(6) CDR1 shown in SEQ ID NO: 2, CDR2 shown in SEQ ID NO: 3, and CDR3 shown in SEQ ID NO: 101;
(7) CDR1 shown in SEQ ID NO: 2, CDR2 shown in SEQ ID NO: 3, and CDR3 shown in SEQ ID NO: 102;
(8) CDR1 shown in SEQ ID NO: 2, CDR2 shown in SEQ ID NO: 3, and CDR3 shown in SEQ ID NO: 103;
(9) CDR1 shown in SEQ ID NO: 2, CDR2 shown in SEQ ID NO: 3, and CDR3 shown in SEQ ID NO: 104;
(10) CDR1 shown in SEQ ID NO: 105, CDR2 shown in SEQ ID NO: 7, and CDR3 shown in SEQ ID NO: 8;
(11) CDR1 shown in SEQ ID NO: 106, CDR2 shown in SEQ ID NO: 7, and CDR3 shown in SEQ ID NO: 8;
(12) CDR1 shown in SEQ ID NO: 107, CDR2 shown in SEQ ID NO: 7, and CDR3 shown in SEQ ID NO: 8;
(13) CDR1 shown in SEQ ID NO: 6, CDR2 shown in SEQ ID NO: 7, and CDR3 shown in SEQ ID NO: 108;
(14) CDR1 shown in SEQ ID NO: 6, CDR2 shown in SEQ ID NO: 7, and CDR3 shown in SEQ ID NO: 109;
(15) CDR1 shown in SEQ ID NO: 6, CDR2 shown in SEQ ID NO: 7, and CDR3 shown in SEQ ID NO: 110;
(16) CDR1 shown in SEQ ID NO: 111, CDR2 shown in SEQ ID NO: 7, and CDR3 shown in SEQ ID NO: 8;
(17) CDR1 shown in SEQ ID NO: 112, CDR2 shown in SEQ ID NO: 7, and CDR3 shown in SEQ ID NO: 8;
(18) CDR1 shown in SEQ ID NO: 6, CDR2 shown in SEQ ID NO: 7, and CDR3 shown in SEQ ID NO: 113;
(19) CDR1 shown in SEQ ID NO: 6, CDR2 shown in SEQ ID NO: 7, and CDR3 shown in SEQ ID NO: 114; or
(20) CDR1 shown in SEQ ID NO: 6, CDR2 shown in SEQ ID NO: 7, and CDR3 shown in SEQ ID NO: 115.

In another preferred embodiment, the nanobody that specifically binds to human PD-L1 comprises a nanobody of human PD-L1 that has a reverse mutation, and the reverse mutation is an amino acid reverse mutation on the VHH shown in SEQ ID NO. 1 selected from the group consisting of: A6IN, A61Q, A61K, D59R, D59M, D59F, D62W, D62Y, C103K, C103M, C103W, G99R, G99Q, G99W, G99Y; or an amino acid reverse mutation on the VHH shown in SEQ ID NO. 5 selected from the group consisting of: G30R, G30F, G30W, G101R, G101W, G101Y, F29R, F29W, P100R, P100Q, P100E; preferably, the reverse mutation is an amino acid reverse mutation on the VHH shown in SEQ ID NO. 1 selected from the group consisting of: C103K, C103M, C103W, G99R, G99Q, G99W, G99Y; or an amino acid reverse mutation on the VHH shown in SEQ ID NO. 5 selected from the group consisting of: G30R, G30F, G30W, G101R, G101W, G101Y, F29R, F29W, P100R, P100Q, P100E.

In another preferred embodiment, the amino acid sequence of the VHH chain of the nanobody that specifically binds to human PD-L1 is selected from the group consisting of:
(a) an amino acid sequence as shown in SEQ ID NOs: 1, 5, 9, 13, 17, 20, 24, 28, 32, 36, 39;
(b) a derivative antibody or an active fragment formed by adding one or more amino acids, substituting one or more amino acids, or deleting 1-3 amino acids in the amino acid sequences of (a), wherein the derivative antibody or active fragment retains the ability to specifically bind to PD-L1.

In another preferred embodiment, the sequence of the nanobody comprises an amino acid sequence having at least 80%, preferably at least 90%, more preferably at least 95%, even more preferably at least 99% homology to SEQ ID NO: 1, 5, 9, 13, 17, 20, 24, 28, 32, 36 or 39.

In another preferred embodiment, the nanobody that specifically binds to human PD-L1 is a humanized nanobody that specifically binds to human PD-L1, and the nanobody comprises a VHH chain, the amino acid sequence of which is selected from the group consisting of:
(a) an amino acid sequence as shown in SEQ ID NOs: 43, 44, 45, 46, 47, 48, 49, 50, 51, 52, 53, 54, 55, 56, 57, 58, 59, 60, 61, 62, 63, 64, 65, 66, 67, 68, 69, 70, 71;
(b) a derivative antibody or an active fragment formed by adding one or more amino acids, substituting one or more amino acids, or deleting 1-3 amino acids in the amino acid sequences of (a), wherein the derivative antibody or active fragment retains the ability to specifically bind to PD-L1.

In another preferred embodiment, the sequence of the nanobody comprises an amino acid sequence having at least 80%, preferably at least 90%, more preferably at least 95%, even more preferably at least 99% homology to SEQ ID NO: 43, 44, 45, 46, 47, 48, 49, 50, 51, 52, 53, 54, 55, 56, 57, 58, 59, 60, 61, 62, 63, 64, 65, 66, 67, 68, 69, 70 or 71.

In another preferred embodiment, the nanobody that specifically binds to human PD-L1 is an affinity-matured nanobody that specifically binds to human PD-L1, and the nanobody comprises a VHH chain, the amino acid sequence of which is selected from the group consisting of:
(a) an amino acid sequence as shown in SEQ ID NO: 72, 73, 74, 75, 76, 77, 78, 79, 80, 81, 82, 83, 84, 85, 86, 87, 88, 89, 90, 91, 92, 93, 94, 95, 96 or 97;
(b) a derivative antibody or an active fragment formed by adding one or more amino acids, substituting one or more amino acids, or deleting 1-3 amino acids in the amino acid sequences of (a), wherein the derivative antibody or active fragment retains the ability to specifically bind to PD-L1.

In another preferred embodiment, the sequence of the nanobody comprises an amino acid sequence having at least 80%, preferably at least 90%, more preferably at least 95%, even more preferably at least 99% homology to SEQ ID NO: 72, 73, 74, 75, 76, 77, 78, 79, 80, 81, 82, 83, 84, 85, 86, 87, 88, 89, 90, 91, 92, 93, 94, 95, 96 or 97.

In another preferred embodiment, the “affinity maturation” means that the affinity of the anti-human PD-L1 nanobody modified by affinity maturation for PD-L1 is increased by at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 20-fold, or at least 25-fold relative to the affinity of the anti-human PD-L1 nanobody for PD-L1 before the modification.

In another preferred embodiment, the complementarity determining region CDR of the VHH chain of the nanobody consists of CDR1 shown in SEQ ID NO: 2, CDR2 shown in SEQ ID NO: 3, and CDR3 shown in SEQ ID NO: 4.

In another preferred embodiment, the VHH chain sequence of the nanobody is as shown in SEQ ID NO.: 1.

In another preferred embodiment, the complementarity determining region CDR of the VHH chain of the nanobody consists of CDR1 shown in SEQ ID NO: 6, CDR2 shown in SEQ ID NO: 7, and CDR3 shown in SEQ ID NO: 8.

In another preferred embodiment, the VHH chain sequence of the nanobody is as shown in SEQ ID NO.: 5.

In another preferred embodiment, the TLR agonist is a macromolecule (protein or nucleic acid) or a small molecule agonist.

In another preferred embodiment, the TLR agonist comprises but is not limited to a TLR1 agonist, a TLR2 agonist, a TLR3 agonist, a TLR4 agonist, a TLR5 agonist, a TLR6 agonist, a TLR7 agonist, a TLR8 agonist and a TLR9 agonist.

In another preferred embodiment, n is the average number of conjugated drugs in the antibody-drug conjugate, preferably n is 1 to 9, preferably 2.5 to 6.5, and more preferably 3.5 to 5.5.

In another preferred embodiment, the TLR agonist is a TLR7 agonist.

In another preferred embodiment, the TLR agonist does not have TLR8 agonistic activity.

In another preferred embodiment, the TLR7 agonist is a host endogenous agonist or an exogenous agonist.

In another preferred embodiment, the TLR7 agonist is a small molecule agonist.

In another preferred embodiment, the TLR7 agonist comprises: SZU-101:

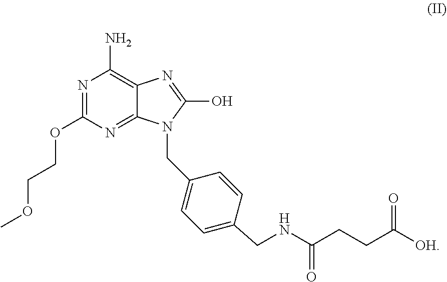 [SMILES: COCCOc1nc(N)c2nc(O)n(Cc3ccc(CNC(=O)CCC(=O)O)cc3)c2n1]

In another preferred embodiment, the TLR7 agonist is a derivative compound of SZU-101, including but not limited to substitution, modification or deletion of one or more groups based on SZU-101.

In another preferred embodiment, the TLR7 agonist is a multivalent compound of SZU-101.

In another preferred embodiment, the TLR7 agonist (such as SZU-101) is linked to the terminal amino group or side chain amino group of the heavy chain constant region or heavy chain variable domain (VHH) of the PD-L1 antibody.

In another preferred embodiment, the TLR7 agonist (such as SZU-101) is linked to the thiol group of the PD-L1 antibody.

In another preferred embodiment, the SZU-101 is linked to the amino group of the PD-L1 antibody to form the structure shown in S1:

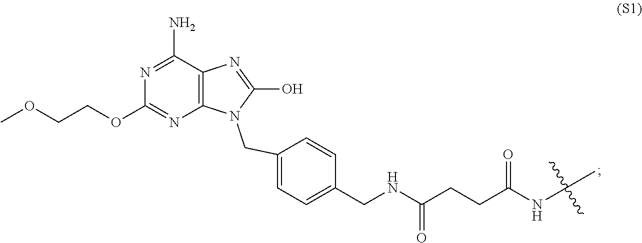 [SMILES: *NC(=O)CCC(=O)NCc1ccc(Cn2c(O)nc3c(N)nc(OCCOC)nc32)cc1]

or
the SZU-101 is linked to the thiol group of the PD-L1 antibody to form the structure shown in S2:

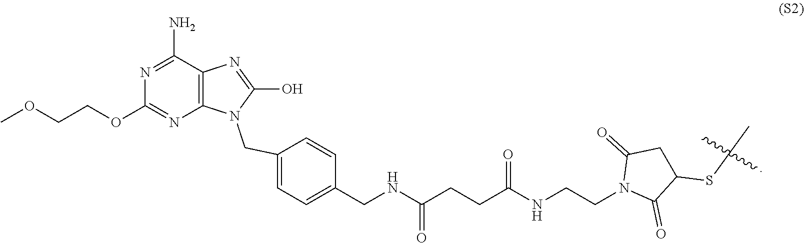 [SMILES: *SC1CC(=O)N(CCNC(=O)CCC(=O)NCc2ccc(Cn3c(O)nc4c(N)nc(OCCOC)nc43)cc2)C1=O]

In another preferred embodiment, the TLR7 agonist is fixed-point and/or randomly linked to the PD-L1 antibody (i.e., in formula I, the U is fixed-point and/or randomly linked to Z).

In another preferred embodiment, the U is linked to Z.

In another preferred embodiment, the U is fixed-point linked to an amino acid site of the PD-L1 antibody Z selected from the group consisting of: G, K, L, A, C or a combination thereof.

In another preferred embodiment, the chemical bond is polyethylene glycol (PEG).

In another preferred embodiment, the chemical bond is a PEG derivative compound, including but not limited to substitution, modification or deletion of one or more groups based on SZU-101.

In another preferred embodiment, the degree of polymerization of the PEG chemical bond is a positive integer greater than or equal to 1.

In another preferred embodiment, the antibody-drug conjugate increases the PD-L1 level of cells within the tumor.

In another preferred embodiment, the antibody-drug conjugate activates immune cells.

In another preferred embodiment, the activation is an in vitro activation.

In another preferred embodiment, the in vitro activation includes: culturing an immune cell for a period of time (e.g., 6-48 hours) in the presence of the antibody-drug conjugate, thereby obtaining an immune cell that has been immune-activated.

In another preferred embodiment, the immune cell is selected from but not limited to: CD8+ T cell, natural killer (NK) cell, dendritic cell, lymphocyte, monocyte/macrophage, granulocyte, or a combination thereof.

In another preferred embodiment, the antibody-drug conjugate or a pharmaceutically acceptable salt thereof is used to prepare a composition or preparation, and the composition or preparation is used for:
(a) promoting the maturation of a dendritic cells;
(b) enhancing the function of tumor-infiltrating cytotoxic cells (CD8+ T cell and NK cell);
(c) promoting the expression of granzyme B and IFN-γ in tumor-infiltrating cytotoxic cells;
(d) promoting the repolarization of tumor-associated macrophages;
(e) reducing infiltration of TGF-β+ macrophages;
(f) promoting the infiltration of IFN-γ+ CD 4+ T cells;
(g) promoting the expression of PD-L1 by intratumoral macrophages;
(h) targeting and reconstituting the tumor immune microenvironment;
(i) increasing the level of PD-L1 in tumor cells; and/or
(j) treating a tumor with a moderate or low PD-L1 expression.

In another preferred embodiment, the reconstitution of the tumor immune microenvironment is to coordinate the anti-tumor immune response of innate immunity and adaptive immunity in a tumor.

In another preferred embodiment, the reconstitution of the tumor immune microenvironment is to increase the infiltration of an anti-tumor immune cell and reduce the proportion of an immunosuppressive cell.

In another preferred embodiment, the anti-tumor immune cell comprises but is not limited to CD8+ T cell and NK cell that secrete granzymes and IFN-γ, activated dendritic cell, CD4+ T cell that secretes IFN-γ, and M1 macrophages.

In another preferred embodiment, the immunosuppressive cell comprises but is not limited to M2 macrophage, Treg cell, and leukocyte secreting TGF-β.

In another preferred embodiment, the PD-L1 level comprises the PD-L1 level on cell surface and the intracellular PD-L1 level.

In another preferred embodiment, the tumor with a low PD-L1 expression is a solid tumor or a hematological tumor.

In the second aspect of the present invention, a PD-L1 nanobody is provided, wherein the PD-L1 nanobody specifically binds to human PD-L1, and the complementarity determining region CDR of the VHH chain in the nanobody is selected from the group consisting of:
(1) CDR1 shown in SEQ ID NO: 2, CDR2 shown in SEQ ID NO: 3, and CDR3 shown in SEQ ID NO: 4;
(2) CDR1 shown in SEQ ID NO: 6, CDR2 shown in SEQ ID NO: 7, and CDR3 shown in SEQ ID NO: 8;
(3) CDR1 shown in SEQ ID NO: 10, CDR2 shown in SEQ ID NO: 11, and CDR3 shown in SEQ ID NO: 12;
(4) CDR1 shown in SEQ ID NO: 14, CDR2 shown in SEQ ID NO: 15, and CDR3 shown in SEQ ID NO: 16;
(5) CDR1 shown in SEQ ID NO: 2, CDR2 shown in SEQ ID NO: 18, and CDR3 shown in SEQ ID NO: 19;
(6) CDR1 shown in SEQ ID NO: 21, CDR2 shown in SEQ ID NO: 22, and CDR3 shown in SEQ ID NO: 23;
(7) CDR1 shown in SEQ ID NO: 25, CDR2 shown in SEQ ID NO: 26, and CDR3 shown in SEQ ID NO: 27;
(8) CDR1 shown in SEQ ID NO: 29, CDR2 shown in SEQ ID NO: 30, and CDR3 shown in SEQ ID NO: 31;
(9) CDR1 shown in SEQ ID NO: 33, CDR2 shown in SEQ ID NO: 34, and CDR3 shown in SEQ ID NO: 35;
(10) CDR1 shown in SEQ ID NO: 37, CDR2 shown in SEQ ID NO: 30, and CDR3 shown in SEQ ID NO: 38;
(11) CDR1 shown in SEQ ID NO: 40, CDR2 shown in SEQ ID NO: 41, and CDR3 shown in SEQ ID NO: 42;

In another preferred embodiment, any one of the above amino acid sequences also comprises a derivative sequence that is optionally added, deleted, modified and/or substituted with at least one (such as 1-3, preferably 1-2, more preferably 1) amino acid and may retain the ability to bind to PD-L1.

In another preferred embodiment, the amino acid sequence of the VHH chain of the nanobody that specifically binds to human PD-L1 is selected from the group consisting of:
(a) an amino acid sequence as shown in SEQ ID NO: 1, 5, 9, 13, 17, 20, 24, 28, 32, 36, or 39;
(b) a derivative antibody or an active fragment formed by adding one or more amino acids, substituting one or more amino acids, or deleting 1-3 amino acids in the amino acid sequences of (a), wherein the derivative antibody or active fragment retains the ability to specifically bind to PD-L1.

In another preferred embodiment, the sequence of the nanobody comprises an amino acid sequence having at least 80%, preferably at least 90%, more preferably at least 95%, even more preferably at least 99% homology to SEQ ID NO: 1, 5, 9, 13, 17, 20, 24, 28, 32, 36 or 39.

In another preferred embodiment, the nanobody that specifically binds to human PD-L1 is a humanized nanobody that specifically binds to human PD-L1, and the nanobody comprises a VHH chain, the amino acid sequence of which is selected from the group consisting of:
(a) an amino acid sequence as shown in SEQ ID NO: 43, 44, 45, 46, 47, 48, 49, 50, 51, 52, 53, 54, 55, 56, 57, 58, 59, 60, 61, 62, 63, 64, 65, 66, 67, 68, 69, 70, 71;
(b) a derivative antibody or an active fragment formed by adding one or more amino acids, substituting one or more amino acids, or deleting 1-3 amino acids in the amino acid sequences of (a), wherein the derivative antibody or active fragment retains the ability to specifically bind to PD-L1.

In another preferred embodiment, the sequence of the nanobody comprises an amino acid sequence having at least 80%, preferably at least 90%, more preferably at least 95%, even more preferably at least 99% homology to SEQ ID NO: 43, 44, 45, 46, 47, 48, 49, 50, 51, 52, 53, 54, 55, 56, 57, 58, 59, 60, 61, 62, 63, 64, 65, 66, 67, 68, 69, 70 or 71.

In another preferred embodiment, the nanobody that specifically binds to human PD-L1 is an affinity-matured nanobody that specifically binds to human PD-L1, and the nanobody comprises a VHH chain, the amino acid sequence of which is selected from the group consisting of:
(a) an amino acid sequence as shown in SEQ ID NO: 72, 73, 74, 75, 76, 77, 78, 79, 80, 81, 82, 83, 84, 85, 86, 87, 88, 89, 90, 91, 92, 93, 94, 95, 96 or 97;
(b) a derivative antibody or an active fragment formed by adding one or more amino acids, substituting one or more amino acids, or deleting 1-3 amino acids in the amino acid sequences of (a), wherein the derivative antibody or active fragment retains the ability to specifically bind to PD-L1.

In another preferred embodiment, the sequence of the nanobody comprises an amino acid sequence having at least 80%, preferably at least 90%, more preferably at least 95%, even more preferably at least 99% homology to SEQ ID NO: 72, 73, 74, 75, 76, 77, 78, 79, 80, 81, 82, 83, 84, 85, 86, 87, 88, 89, 90, 91, 92, 93, 94, 95, 96 or 97.

In the third aspect of the present invention, a pharmaceutical composition is provided, wherein the pharmaceutical composition comprises:
(a) the antibody-drug conjugate or a pharmaceutically acceptable salt thereof of the first aspect of the invention, or the PD-L1 nanobody of the second aspect of the invention; and
(b) a pharmaceutically acceptable carrier.

In another preferred embodiment, the pharmaceutical composition further comprises:
(c) other biologically active drugs, such as drugs for treating tumors.

In another preferred embodiment, the other biologically active drugs promote the anti-tumor function of CD8+ T cells and NK cells.

In another preferred embodiment, the pharmaceutical composition comprises a single drug, a compound drug, or a synergistic drug.

In another preferred embodiment, the administration method for the pharmaceutical composition is selected from the group consisting of: subcutaneous injection, intradermal injection, intramuscular injection, intravenous injection, intraperitoneal injection, microneedle injection, oral administration, or oral and nasal spraying and aerosol inhalation.

In another preferred embodiment, the administration method for the pharmaceutical composition is by co-culturing the pharmaceutical composition and an immune cell (such as dendritic cell, natural killer cell, lymphocyte, monocyte/macrophage, granulocyte, etc.), and then separating the immune cells for in vivo reinfusion.

In another preferred embodiment, the dosage form of the pharmaceutical composition is selected from the group consisting of: a liquid, solid, or gel.

In another preferred embodiment, the pharmaceutical composition is used for anti-tumor treatment.

In another preferred embodiment, the pharmaceutical composition is used to treat a tumor with a low expression of PD-L1.

In another preferred embodiment, “low expression of PD-L1” means that the amount E1 of PD-L1 expressed by the tumor is lower than the amount E0 of PD-L1 expressed by a normal tumor, preferably E1/E0≤1/2, more preferably ≤1/3, and further preferably ≤1/4.

In another preferred embodiment, the tumor comprises but is not limited to: a breast cancer, liver cancer, gastric cancer, colon cancer, leukemia, lung cancer, kidney tumor, small intestine cancer, prostate cancer, colorectal cancer, prostate cancer, cervical cancer, lymphoma, bone cancer, adrenal tumor, or bladder tumor.

In the fourth aspect of the present invention, an immunoconjugate is provided, wherein the immunoconjugate comprises:
(a) the antibody-drug conjugate according to the first aspect of the present invention; and
(b) another coupling moiety.

In another preferred embodiment, the coupling moiety is selected from the group consisting of: a small molecule compound, PEG, fluorescein, radioisotope, contrast agent, fatty acid chain, protein fragment, or a combination thereof.

In another preferred embodiment, the components (a) and (b) are operably linked.

In another preferred embodiment, the coupling moiety comprises a chemical label and a biological label.

In another preferred embodiment, the chemical label is selected from an isotope, immunotoxin and/or chemical drug.

In another preferred embodiment, the biomarker is selected from a biotin, avidin or enzyme label.

In another preferred embodiment, the small molecule compound is selected from a drug or a toxin for treating a tumor or an autoimmune disease.

In another preferred embodiment, the radioisotope comprises:
(i) a diagnostic isotope selected from the group consisting of Tc-99m, Ga-68, F-18, I-123, I-125, I-131, In-111, Ga-67, Cu-64, Zr-89, C-11, Lu-177, Re-188, or a combination thereof; and/or
(ii) a therapeutic isotope, wherein the therapeutic isotope is selected from the group consisting of Lu-177, Y-90, Ac-225, As-211, Bi-212, Bi-213, Cs-137, Cr-51, Co-60, Dy-165, Er-169, Fm-255, Au-198, Ho-166, I-125, I-131, Ir-192, Fe-59, Pb-212, Mo-99, Pd-103, P-32, K-42, Re-186, Re-188, Sm-153, Ra223, Ru-106, Na24, Sr89, Tb-149, Th-227, Xe-133, Yb-169, Yb-177, or a combination thereof.

In another preferred embodiment, the radioisotope comprises but is not limited to iodine 131, indium 111 and lutetium 177.

In another preferred embodiment, the contrast agent is used for MRI or CT.

In another preferred embodiment, the protein fragment comprises but is not limited to antibody Fc, biotin, avidin, HRP, antibody, enzyme, cytokine and other biologically active proteins or polypeptides.

In another preferred embodiment, the coupling moiety is a detectable label.

In another preferred embodiment, the coupling moiety is selected from the group consisting of a fluorescent or luminescent marker, radioactive marker, MRI (magnetic resonance imaging) or CT (computer X-ray tomography) contrast agent, or enzyme capable of producing a detectable product, radionuclide, biotoxin, cytokine (such as IL-2, etc.), antibody, antibody Fc fragment, antibody scFv fragment, gold nanoparticle/nanorod, viral particle, liposome, nanomagnetic particle, prodrug-activating enzyme (for example, DT-diaphorase (DTD) or biphenyl hydrolase-like protein (BPHL)) or any form of nanoparticles.

In the fifth aspect of the present invention, a fusion protein is provided, wherein the fusion protein comprises:
(a) the PD-L1 nanobody of the second aspect of the present invention; and
(b) optionally a polypeptide molecule or protein fragment having a therapeutic function.

In another preferred embodiment, the polypeptide molecule or fragment having a therapeutic function comprises but is not limited to: a polypeptide molecule or a fragment targeting PD-1, IL-4R, IL-4Ra, TNF-α, VEGF, 4-1BB, CD47, TIM3, CTLA4, IL-17A, CD19, CD22, CD28, CD38, CD40, CD47, B7-H3, TSLP, BCMA, GLP-1, Trop2, TIGIT, LAG-3, FGL1, and HER2.

In another preferred embodiment, the polypeptide molecule or fragment having a therapeutic function comprises, but is not limited to, insulin, IL-2, interferon, calcitonin, GHRH peptide, intestinal peptide analog, albumin, antibody fragment, and cytokine.

In another preferred embodiment, the polypeptide molecule or fragment having a therapeutic function comprises a single-chain antibody (scFv), a double-chain antibody, a monoclonal antibody, or a chimeric antibody.

In another preferred embodiment, the fusion protein further comprises a tag sequence for facilitating expression and/or purification.

In another preferred embodiment, the tag sequence is selected from the group consisting of: a 6His tag, GGGS sequence, FLAG tag.

In another preferred embodiment, the fusion protein comprises a bispecific antibody or a chimeric antibody.

In the sixth aspect of the present invention, a multispecific antibody is provided, wherein the multispecific antibody comprises:
(a) the PD-L1 nanobody of the second aspect of the present invention; and
(b) optionally an antibody molecule targeting a second antigen.

In another preferred embodiment, the multispecific antibody further comprises a second antigen binding region targeting a target selected from the group consisting of PD-1, IL-4R, IL-4Ra, TNF-α, VEGF, 4-1BB, CD47, TIM3, CTLA4, IL-17A, CD19, CD22, CD28, CD38, CD40, CD47, B7-H3, TSLP, BCMA, GLP-1, Trop2, TIGIT, LAG-3, FGL1, HER2 or a combination thereof.

In another preferred embodiment, the second antigen binding region is a nanobody.

In another preferred embodiment, the multispecific antibody comprises one or more second antigen binding regions.

In another preferred embodiment, the multispecific antibody further comprises an antibody Fc segment.

In the seventh aspect of the present invention, it provides a method for preparing the antibody-drug conjugate of the first aspect of the present invention, wherein the method comprises steps of:

preparing a reaction system, wherein the reaction system comprises an antibody and a free drug molecule, and then conducting a coupling reaction to obtain the antibody-drug conjugate, wherein the drug molecule comprises a TLR agonist and a linker.

In another preferred embodiment, the reaction time is 3 h-10 h.

In another preferred embodiment, the molar ratio of the antibody to the drug molecule is 1-2:3-20; preferably 1:6-10.

In another preferred embodiment, SZU-101, EDCI and NHS are dissolved in DMSO and stirred at room temperature for three hours to prepare SZU-101-NHS active ester, and the PD-L1 nanobody and SZU-101-NHS active ester are stirred at 4° C. for 4 hours at a molar ratio of 1:10 to prepare a nanobody-drug conjugate.

In the eighth aspect of the present invention, a medical kit is provided, wherein the medical kit comprises:
(1) a first container, in which the PD-L1 nanobody of the second aspect of the present invention, and a pharmaceutically acceptable carrier are contained;
(2) a second container, in which a TLR7 agonist, and a pharmaceutically acceptable carrier are contained;
and (3) optionally an instruction manual.

In the ninth aspect of the present invention, it provides a method for preventing or treating a tumor, wherein the method comprises administering to a subject in need thereof the nanobody-drug conjugate of the first aspect of the present invention, the PD-L1 nanobody of the second aspect of the present invention, or the medical kit of the eighth aspect of the present invention.

In another preferred embodiment, the tumor is a tumor expressing PD-L1.

In another preferred embodiment, the tumor is selected from the group consisting of: a tumor with a high expression of PD-L1, a tumor with a medium expression of PD-L1, and a tumor with a low expression of PD-L1.

In another preferred embodiment, the tumor is a tumor with a medium expression of PD-L1 or a tumor with a low expression of PD-L1.

In another preferred embodiment, the tumor is a tumor with a low expression of PD-L1.

In another preferred embodiment, “high expression of PD-L1” means that E1/E0, the ratio of E1 (the amount of PD-L1 expressed by the tumor) to E0 (the amount of PD-L1 expressed by a normal tumor) is greater than 1, preferably ≥1.5, and more preferably ≥2.0.

In another preferred embodiment, “medium expression of PD-L1” means that E1/E0, the ratio of E1 (the amount of PD-L1 expressed by the tumor) to E0 (the amount of PD-L1 expressed by a normal tumor) is 0.5-1.1, more preferably 0.7-1.0, and more preferably 0.8-0.9.

In another preferred embodiment, “low expression of PD-L1” means that E1/E0, the ratio of E1 (the amount of PD-L1 expressed by the tumor) to E0 (the amount of PD-L1 expressed by a normal tumor) is ≤1/2, more preferably ≤1/3, and more preferably ≤1/4.

In another preferred embodiment, the tumor comprises but is not limited to: a breast cancer, liver cancer, gastric cancer, colon cancer, leukemia, lung cancer, kidney tumor, small intestine cancer, prostate cancer, colorectal cancer, prostate cancer, cervical cancer, lymphoma, bone cancer, adrenal tumor, or bladder tumor.

In the tenth aspect of the present invention, it provides a use of an antibody-drug conjugate of the first aspect of the present invention, the PD-L1 nanobody of the second aspect of the present invention, the pharmaceutical composition of the third aspect of the present invention, the immunoconjugate of the fourth aspect of the present invention, the fusion protein of the fifth aspect of the present invention, or the multispecific antibody of the sixth aspect of the present invention in the manufacture of a reagent, a detection plate or a kit; or in the manufacture of a medicament for preventing and/or treating a PD-L1 related disease.

In another preferred embodiment, the PD-L1 related disease is a tumor/cancer expressing PD-L1.

It should be understood that within the scope of the present invention, each technical feature of the present invention described above and in the following (such as examples) may be combined with each other to form a new or preferred technical solution, which is not listed here due to space limitations.

### DESCRIPTION OF THE DRAWINGS

FIG. 1 shows the binding activity to human PD-L1 of the candidate anti-human PD-L1 nanobodies, wherein the blank is a negative control.

FIG. 2 shows the assay of activity of candidate anti-human PD-L1 nanobodies in blocking human PD-1/PD-L1 binding, wherein the blank and blank+ligand are control groups.

FIG. 3 shows the IC₅₀ curve of the candidate anti-human PD-L1 nanobodies in blocking human PD-1/PD-L1 binding, wherein KN035 is a positive control.

FIG. 4 shows the mass spectrometric identification of the candidate anti-human PD-L1 nanobody-drug conjugate before and after conjugation (4A-D) and the mass spectrometric identification of the positive control group KN035 (4E).

FIG. 5 shows the tumor inhibitory effects of candidate anti-human PD-L1 nanobodies h_Nb1, h_Nb2, positive control antibody KN035, and nanobody-drug conjugates h_Nb1-SZU-101 and h_Nb2-SZU-101. 5A is the tumor growth curve of different groups, 5B is the endpoint tumor weight of each group; 5C is the tumor inhibition rate of each group, 5D-51 are the tumor growth curves of each mouse in different groups, and 5J-5K are the tumor growth curves of tumor regression mice after administrating the nanobody-drug conjugates h_Nb1-SZU-101 and h_Nb2-SZU-101 in tumor-rebearing experiment.

### DETAILED DESCRIPTION

After extensive and in-depth research, the inventors screened and identified an anti-human PD-L1 nanobody, and developed an anti-human PD-L1 and TLR7 dual-targeting nanobody-drug conjugate. Specifically, in the present invention, phage display technology and flow cytometry are used to screen and obtain anti-human PD-L1 nanobodies and derivative molecules thereof, and antibody conjugation technology is used to prepare and obtain new dual-targeting nanobody-drug conjugates and derivative molecules thereof, and anti-human PD-L1 nanobodies and dual-targeting nanobody-drug conjugates having excellent anti-tumor activity are found. In addition, it is found, in the present invention, that dual-targeting nanobody-drug conjugates may reconstitute the tumor immune microenvironment, promote antigen-presenting cells including macrophages to express PD-L1, and are beneficial for the treatment of “cold” tumors with low expression of PD-L1 molecules. The PD-L1 and TLR7 dual-targeting nanobody-drug conjugate of the present invention shows outstanding anti-tumor effects and novel mechanisms of action, and have clinical development and application value, on which the present invention was completed.

### TLR Receptors and TLR Receptor Agonists

As used herein, the term “TLR receptor” refers to a Toll-like receptor, which is an important class of innate immune pattern recognition receptors in the immune system of organisms, which may specifically recognize relatively conservative antigen molecules (or pathogen-associated molecular patterns) in the evolution of pathogenic microorganisms, and achieve effective detection of pathogenic microorganism invasion and induction of innate immune responses. 10 TLR receptors have been found in the human body, namely TLR1-TLR10, wherein TLR3, TLR7, TLR8, and TLR9 are located on the endosome and lysosome membranes of cells, and the rest are located on the cytoplasmic membrane. In an embodiment of the present invention, TLR7 is preferably used as one of the drug molecular targets. The natural ligand of the TLR7 molecule is a single-stranded linear RNA.

As used herein, the term “TLR receptor agonist” refers to a macromolecule (protein or nucleic acid) or small molecule agonist that may specifically bind to and activate TLR receptors, promote the transduction of downstream signals of TLR receptors, and achieve the activation of innate immune cells. In an embodiment of the present invention, a TLR7 agonist is preferably used to construct a nanobody drug conjugate. In addition to SZU-101 used in an embodiment of the present invention, available TLR7 agonists also include imiquimod and R848, etc.

As used herein, the terms “nanobody of the present invention”, “PD-L1 targeting nanobody of the present invention”, and “anti-PD-L1 nanobody of the present invention” are used interchangeably and refer to a nanobody that specifically recognizes and binds to PD-L1 (including human or mouse PD-L1). Particularly preferred is a nanobody (h_Nb1 or h_Nb2), VHH chain amino acid sequence of which is shown in SEQ ID NO: 1 or SEQ ID NO: 5.

As used herein, the terms “nanobody drug conjugate of the present invention”, “dual-targeting nanobody drug conjugate of the present invention”, and “PD-L1 and TLR7 dual-targeting nanobody drug conjugate of the present invention” are used interchangeably, and all refer to a novel drug molecule formed by a nanobody and its derivative protein conjugated to a TLR7 agonist that specifically recognizes and binds to PD-L1 (including human or mouse PD-L1). The nanobody in the nanobody-drug conjugate is particularly preferably a nanobody, the VHH chain amino acid sequence of which is shown in SEQ ID NO: 1 or SEQ ID NO: 2.

As used herein, the term “antibody” or “immunoglobulin” is a heterotetrameric glycoprotein of about 150,000 daltons with identical structural features, which consists of two identical light chains (L) and two identical heavy chains (H). Each light chain is linked to a heavy chain via a covalent disulfide bond, and different immunoglobulin isotypes have different numbers of disulfide bonds between the heavy chains. There are also regularly spaced intrachain disulfide bonds in each heavy and each light chain. Each heavy chain has a variable region (VH) at one end, followed by a plurality of constant regions. Each light chain has a variable region (VL) at one end and a constant region at the other end; the constant region of light chain pairs with the first constant region of heavy chain, and the variable region of light chain pairs with the variable region of heavy chain. Special amino acid residues form an interface between the variable regions of a light chain and a heavy chain.

As used herein, the terms “single domain antibody (sdAb, or VHH)” and “nanobody” have the same meaning, referring to cloning the variable region of the heavy chain of an antibody constructing a nanobody composed of only one heavy chain variable region, which is the smallest antigen-binding fragment with complete function. Usually, an antibody naturally lacking the light chain and heavy chain constant region 1 (CH1) is first obtained, and then the variable region of the antibody heavy chain is cloned to construct a nanobody (VHH) composed of only one heavy chain variable region.

Nanobody is a new type of small molecule antibody fragment, which is cloned from the variable region (VHH) of the heavy chain antibody of camel. Nanobody (Nb) has excellent biological properties, with a molecular weight of 12-15 kDa, which is one-tenth of the complete antibody. It has good tissue penetration, high specificity and good water solubility. Due to its special structural properties, it has the advantages of both traditional antibodies and small molecule drugs, and almost perfectly overcomes the defects of traditional antibodies such as long development cycle, low stability and harsh storage conditions. It has gradually become an emerging force in the new generation of antibody therapy, showing broad application prospects in immune diagnosis and treatment.

As used herein, the term “variable” means that certain portion of the variable region in an antibody differ in sequence, which is responsible for the binding and specificity of various specific antibodies to their specific antigen. However, the variability is not distributed evenly throughout the variable regions of an antibody. It is concentrated in three fragments called complementarity determination regions (CDRs) or hypervariable regions in light chain and heavy chain variable regions. The conserved parts of variable regions are called framework regions (FRs). Each of the variable regions of naturally occurring heavy and light chains comprises four FR regions, which are generally in a β-sheet configuration, joined by the three CDRs forming a linking loop, and in some cases, may form a partical β-sheet structure. The CDRs in each chain are closely linked together via the FR regions, and together with the CDRs of the other chain, form the antigen binding site of an antibody (see Kabat et al., NIH Publ. No. 91-3242, Volume I, pages 647-669 (1991)). Constant regions are not directly involved in the binding of antibodies to antigen, however, they exhibit different effector functions, such as participating in the antibody-dependent cytotoxicity of antibodies.

As known to those skilled in the art, an immunoconjugates and the fusion expression product include: a drug, a toxin, a cytokine, a radionuclide, an enzyme and other diagnostic or therapeutic molecules that bind to the antibody or fragment thereof of the present invention to form a conjugate.

As used herein, the terms “nanobody conjugate drug” and “nanobody drug conjugate” are used interchangeably. As known to those skilled in the art, nanobody conjugate is a special form of antibody-drug conjugates, which are drug molecules formed by conjugating a nanobody or derived protein to a drug, toxin, cytokine, radionuclide, enzyme and other diagnostic or therapeutic molecules, and may be used for tumor treatment, drug delivery and in vivo imaging, etc., which have broad clinical application value.

As used herein, the term “heavy chain variable region” is used interchangeably with “V_{H}”

As used herein, the terms “variable region” and “complementarity determining region (CDR)” are used interchangeably.

In a preferred embodiment of the present invention, the heavy chain variable region of the antibody includes three complementarity determining regions CDR1, CDR2, and CDR3.

In a preferred embodiment of the present invention, the heavy chain of the antibody includes the heavy chain variable region and heavy chain constant region as described above.

In the present invention, the terms “antibody of the present invention”, “protein of the present invention”, or “polypeptide of the present invention” are used interchangeably, and refer to a polypeptide that specifically binds to PD-L1, such as a protein or polypeptide having a heavy chain variable region. They may or may not contain a starting methionine.

The invention also provides other proteins or fusion expression products having the antibody of the present invention. Specifically, the present invention includes any protein or protein conjugate and fusion expression product (i.e., immunoconjugate and fusion expression product) having a heavy chain containing variable regions, as long as the variable region is identical to or has at least 90% homology, preferably at least 95% homology with the variable regions of the heavy chain of the antibody of the present invention.

In general, the antigen binding characteristics of an antibody may be described by three specific regions located in the heavy chain variable region, called the variable region (CDR), which separate the heavy chain variable region into four frame regions (FR). The amino acid sequence of the four FRs is relatively conservative and does not directly participate in the binding reaction. These CDRs form a loop structure, and the β-sheets formed by the FRs in between are spatially close to each other, and the CDRs on the heavy chain and the CDRs on the corresponding light chain constitute the antigen-binding sites of the antibody. It may be determined which amino acids constitute the FR or CDR region by comparing the amino acid sequences of antibodies of the same type.

The variable regions of the heavy chains of the antibody of the present invention are of particular interest because at least part of them involve binding antigens. Therefore, the present invention includes those molecules with a CDR-bearing antibody heavy chain variable region, as long as their CDR has more than 90% (preferably more than 95%, most preferably more than 98%) homology with the CDR identified herein.

The present invention includes not only an intact antibody, but also a fragment of immunologically active antibody or fusion protein formed by an antibody with other sequences. Thus, the present invention also includes a fragment, derivative and analog of the antibody.

As used herein, the terms “fragment”, “derivative” and “analog” refer to a polypeptide that substantially retains the same biological function or activity of the antibody of the present invention. The polypeptide fragment, derivative or analog of the present invention may be (i) a polypeptide with one or more conservative or non-conservative amino acid residues (preferably conservative amino acid residues) substituted, and such substituted amino acid residues may or may not be encoded by the genetic code, or (ii) a polypeptide with a substituent group in one or more amino acid residues, or (iii) a polypeptide formed by fusion of a mature polypeptide with another compound (such as a compound that extends the half-life of the polypeptide, such as polyethylene glycol), or (iv) a polypeptide formed by fusion of an additional amino acid sequence to the polypeptide sequence (such as a leader sequence or secretory sequence or sequence or protein sequence used to purify the polypeptide, or a fusion protein formed with a 6His tag). According to the teachings herein, these fragments, derivatives and analogs are within the scope of well-known to those skilled in the art.

The antibody of the present invention refers to a polypeptide having PD-L1 binding activity and comprising the CDR regions as described above. The term also comprises variant forms of polypeptides having the same function as the antibody of the present invention and comprising the CDR regions as described above. These variants include (but are not limited to): deletion, insertion and/or substitution of one or more (usually 1-50, preferably 1-30, more preferably 1-20, most preferably 1-10) amino acids, and addition of one or more (usually within 20, preferably within 10, more preferably within 5) amino acids at the C-terminal and/or N-terminal. For example, in the art, substitutions with amino acids of similar properties generally do not alter the function of the protein. For another example, addition of one or more amino acids to the C-terminal and/or N-terminal usually does not alter the function of the protein. The term also includes active fragments and active derivatives of the antibody of the present invention.

The variant forms of the polypeptide include homologous sequences, conservative variants, alleles, natural mutants, induced mutants, proteins encoded by DNA capable of hybridizing with the coding DNA of the antibody of the present invention under high or low tightness conditions, and polypeptides or proteins obtained by using anti-serum against the antibody of the present invention.

The present invention also provides other polypeptides, such as a fusion protein containing a nanobody or a fragment thereof. In addition to the almost full-length polypeptide, the present invention also includes a fragment of the nanobody of the present invention. Typically, the fragment has at least about 50 contiguous amino acids, preferably at least about 50 contiguous amino acids, more preferably at least about 80 contiguous amino acids, and most preferably at least about 100 contiguous amino acids of the antibody of the present invention.

In the present invention, “conservative variant of the antibody of the present invention” refers to a polypeptide formed by replacing at most 10, preferably at most 8, more preferably at most 5, and most preferably at most 3 amino acids with amino acids of similar properties as compared with the amino acid sequence of the antibody of the present invention. These conservative variant polypeptides are best produced by amino acid substitution according to Table A.

TABLE A

| | | Preferred |
|---|---|---|
| Initial residue | Representative substitution | substitution |
| Ala (A) | Val; Leu; Ile | Val |
| Arg (R) | Lys; Gln; Asn | Lys |
| Asn(N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The present invention also provides a polynucleotide molecule encoding the above antibody or fragment thereof or fusion protein thereof. The polynucleotide of the present invention may be in the form of DNA or RNA. DNA form includes cDNA, genomic DNA, or synthetic DNA. DNA may be single-stranded or double-stranded. DNA may be a coding strand or a non-coding strand.

The polynucleotide encoding the mature polypeptide of the present invention includes: the coding sequence that encodes only the mature polypeptide; the coding sequence of the mature polypeptide and various additional coding sequences; the coding sequence of the mature polypeptide (and optional additional coding sequence) and the non-coding sequence.

The term “polynucleotide encoding a polypeptide” may be a polynucleotide that includes sequence encoding the polypeptide, or a polynucleotide that also includes additional coding and/or non-coding sequences.

The present invention also relates to a polynucleotide that hybridizes to the sequence as described above and has at least 50%, preferably at least 70%, and more preferably at least 80% identity between the two sequences. In particular, the present invention relates to a polynucleotide that is hybridizable to the polynucleotide of the present invention under strict conditions. In the present invention, “strict condition” refers: (1) hybridization and elution at lower ionic strength and higher temperature, such as 0.2×SSC, 0.1% SDS, 60° C.; or (2) hybridization with denaturing agent, such as 50% (v/v) formamide, 0.1% calf serum/0.1% Ficoll, 42° C., etc.; or (3) hybridization occurs only when the identity between the two sequences is at least 90% or more, more preferably 95% or more. Furthermore, the polypeptide encoded by the hybridizable polynucleotide has the same biological function and activity as the mature polypeptide.

The full-length nucleotide sequence or fragments of the antibody of the present invention may generally be obtained by PCR amplification, recombination or artificial synthesis methods. A feasible method is to synthesize the relevant sequence by artificial synthesis, especially when the fragment length is short. Generally, fragments with a long sequence may be obtained by first synthesizing multiple small fragments followed by ligation. In addition, the coding sequence of the heavy chain and the expression tag (such as 6His) may be fused together to form a fusion protein.

Once the relevant sequence is obtained, the recombination method may be used to obtain the relevant sequence in large quantities. This is usually to clone it into a vector, then transfer it into a cell, and then separate the relevant sequence from the proliferated host cell by conventional methods. The biomolecules (nucleic acids, proteins, etc.) involved in the present invention include biomolecules in isolated form.

At present, the DNA sequence encoding the protein (or a fragment, or a derivative thereof) of the present invention may be obtained completely by chemical synthesis. The DNA sequence may then be introduced into various existing DNA molecules (or, for example, vectors) and cells known in the art. In addition, mutations may be introduced into the protein sequence of the present invention by chemical synthesis.

The present invention also relates to a vector comprising the appropriate DNA sequence as described above and an appropriate promoter or control sequence. These vectors may be used to transform appropriate host cells to enable them to express proteins.

Host cells may be prokaryotic cells, such as bacterial cells; or lower eukaryotic cells, such as yeast cells; or higher eukaryotic cells, such as mammalian cells. Representative examples include: Escherichia coli, Streptomyces; bacterial cells of Salmonella typhimurium; fungal cells such as yeast; insect cells of Drosophila S2 or Sf9; animal cells of CHO, COS7, 293 cells, etc.

Transformation of host cells with recombinant DNA may be carried out using conventional techniques well known to those skilled in the art. When the host is a prokaryotic organism such as Escherichia coli, the competent cells capable of absorbing DNA may be harvested after the exponential growth period and treated with CaCl₂), wherein the steps used are well known in the art. Another method is to use MgCl2. If necessary, the transformation may also be carried out by electroporation. When the host is eukaryotic, the following DNA transfection methods may be used: calcium phosphate co-precipitation method, conventional mechanical methods such as microinjection, electroporation, liposome packaging, etc.

The obtained transformant may be cultured by conventional methods to express the polypeptide encoded by the gene of the present invention. Depending on the host cell used, the medium used in the culture may be selected from a variety of conventional medium. Culture is carried out under conditions suitable for host cell growth. When the host cells grow to an appropriate cell density, the selected promoter is induced by a suitable method (such as temperature conversion or chemical induction), and the cells are cultured for a period of time.

The recombinant polypeptide in the above method may be expressed in the cell, or on the cell membrane, or secreted outside the cell. If necessary, the recombinant protein may be isolated and purified by various separation methods using its physical, chemical and other properties. These methods are well known to those skilled in the art. Examples of these methods include, but are not limited to, conventional renaturation treatment, treatment with a protein precipitant (salting-out method), centrifugation, osmotic breakage, ultra-treatment, ultra-centrifugation, molecular sieve chromatography (gel filtration), adsorption chromatography, ion exchange chromatography, high performance liquid chromatography (HPLC) and other liquid chromatography techniques and combinations of these methods.

The antibody of the present invention may be used alone, or may be combined or coupled with a detectable label (for diagnostic purposes), a therapeutic agent, a PK (protein kinase) modifying moietiy, or any combination of these substances.

A detectable marker for diagnostic purposes includes, but is not limited to, a fluorescent or luminescent label, a radioactive label, a MRI (magnetic resonance imaging) or CT (electronic computer tomography) contrast agent, or an enzyme capable of producing a detectable product.

A therapeutic agent that may bind or couple with the antibody of the present invention includes, but is not limited: 1. a radionuclide; 2. a biological toxin; 3. A cytokine such as IL-2, etc; 4. a gold nanoparticle/nanorod; 5. a viral particle; 6. a liposome; 7. a nanomagnetic particle; 8. a prodrug-activating enzyme (e. g., DT-myoflavase (DTD) or biphenyl hydrolase-like protein (BPHL)).

### Antibody-Drug Conjugates (ADCs)

The present invention further provides an antibody-drug conjugate (ADC) based on the antibody of the present invention.

Typically, the antibody-drug conjugate comprises the antibody and an effector molecule, wherein the antibody is conjugated to the effector molecule, and preferably chemically conjugated, wherein the effector molecule is preferably a drug with therapeutic activity or a drug with immune-promoting function.

The antibody of the present invention and the effector molecule may be conjugated via a coupling agent. Examples of the coupling agent may be any one or more of a non-selective coupling agent, a coupling agent utilizing a carboxyl group, a peptide chain, and a coupling agent utilizing a disulfide bond. The non-selective coupling agent refers to a compound that forms a covalent bond between the effector molecule and the antibody, such as glutaraldehyde. The coupling agent utilizing a carboxyl group may be any one or more of a cis-aconitic anhydride coupling agent (such as cis-aconitic anhydride) and an acylhydrazone coupling agent (the coupling site is an acylhydrazone).

Certain residues on an antibody (such as Cys or Lys, etc.) are used to connect to a variety of functional groups, including imaging agents (such as chromophores and fluorescent groups), diagnostic agents (such as MRI contrast agents and radioisotopes), stabilizers (such as ethylene glycol polymers) and therapeutic agents. An antibody may be coupled to functional agents to form antibody-functional agent conjugates. Functional agents (such as drugs, detection agents, stabilizers) are coupled (covalently linked) to antibodies. Functional agents may be directly or indirectly linked to an antibody through linkers.

An antibody may be coupled to a drug to form antibody-drug conjugates (ADCs). Typically, ADCs contain a linker between the drug and the antibody. The linker may be a cleavable or uncleavable linker. Cleavable linkers are typically easily degraded in the intracellular environment, such as degradation of the linker at the target site, thereby releasing the drug from the antibody. Suitable cleavable linkers include, for example, enzyme-cleavable linkers, including linkers containing peptidyl that may be degraded by intracellular proteases (such as lysosomal proteases or endosomal proteases), or sugar linkers, such as glucuronide-containing linkers that may be degraded by glucuronidase. Peptide linkers may include, for example, dipeptides, such as valine-citrulline, phenylalanine-lysine or valine-alanine. Other suitable cleavable linkers include, for example, pH-sensitive linkers (such as linkers that hydrolyze at pH less than 5.5, such as hydrazone linkers) and linkers that degrade under reducing conditions (such as disulfide linkers). Uncleavable linkers typically release the drug under conditions where the antibody is hydrolyzed by proteases.

Prior to attachment to an antibody, the linker has an active reactive group capable of reacting with certain amino acid residues, and conjugation is achieved through the active reactive group. Thiol-specific active reactive groups are preferred and include, for example, maleimide compounds, halogenated amides (e.g., iodinated, brominated or chlorinated); halogenated esters (e.g., iodinated, brominated or chlorinated); halogenated methyl ketones (e.g., iodinated, brominated or chlorinated), benzyl halides (e.g., iodinated, brominated or chlorinated); vinyl sulfones, pyridyl disulfides; mercury derivatives such 1 as 3,6-di-(mercurymethyl) dioxane, where the counter ion is acetate, chloride or nitrate; and polymethylene dimethyl sulfide thiosulfonate. The linker may include, for example, maleimide linked to the antibody via thiosuccinimide.

It should be understood that the drug may generally be any cytotoxic, cell growth inhibiting or immunosuppressive drug. In the present invention, the drug is a drug that activates or promotes an immune response, such as activating an innate immune response to assist in the activation of adaptive immunity. In a specific embodiment, the drug is a TLR receptor agonist.

In an embodiment, a linker links an antibody and a drug, wherein the drug contains a functional group that may form a bond with the linker. For example, the drug may have an amino group, a carboxyl group, a sulfhydryl group, a hydroxyl group, or a keto group that may form a bond with the linker. In the case where the drug is directly linked to a linker, the drug has a reactive group before being linked to the antibody.

Useful drug categories include, for example, TLR1 agonists, TLR2 agonists, TLR3 agonists, TLR4 agonists, TLR5 agonists, TLR6 agonists, TLR7 agonists, TLR8 agonists and TLR9 agonists, such as SZU-101, imiquimod, R848, CpG, etc. In the present invention, drug-linkers may be used to form an ADC in one simple step. In other embodiments, bifunctional linker compounds may be used to form an ADC in a two-step or multi-step process. For example, a cysteine residue reacts with a reactive portion of a linker in a first step, and in a subsequent step, a functional group on the linker reacts with a drug to form an ADC.

Typically, a functional group on the linker is selected to react specifically with an appropriate reactive group on the drug moiety. As a non-limiting example, an azide-based moiety may be used to react specifically with a reactive alkynyl group on the drug moiety. The drug is covalently attached to the linker via 1,3-dipolar cycloaddition between the azide and the alkynyl group. Other useful functional groups include, for example, ketones and aldehydes (suitable for reaction with hydrazides and alkoxyamines), phosphines (suitable for reaction with azides); isocyanates and isothiocyanates (suitable for reaction with amines and alcohols); and activated esters, such as N-hydroxysuccinimide esters (suitable for reaction with amines and alcohols). These and other attachment strategies, such as those described in Bioconjugation Technology, 2nd Edition (Elsevier), are well known to those skilled in the art. Those skilled in the art will understand that when a complementary pair of reactive functional groups is selected for selective reaction of the drug moiety and the linker, each member of the complementary pair may be used for both the linker and the drug.

The present invention also provides a method for preparing ADC, which may further include: combining an antibody with a drug-linker compound under conditions sufficient to form an antibody conjugate (ADC).

In certain embodiments, the methods of the invention include: combining an antibody with a bifunctional linker compound under conditions sufficient to form an antibody-linker conjugate. In these embodiments, the methods of the invention further include: combining an antibody-linker conjugate with a drug moiety under conditions sufficient for the drug moiety to covalently link to the antibody via a linker.

In some embodiments, the antibody drug conjugate ADC has the following molecular formula:

$$Ab - {( J - U )}_{n} \qquad ( I )$$

wherein,
Ab is a PD-L1 antibody;
Each U is independently a TLR agonist;
J is a chemical bond or linker;
n is 0 or a positive integer; and
“—” is a chemical bond, a connector or a linker.

### USE

The present invention provides uses of the antibodies of the present invention, for example, for manufacture of a diagnostic preparation, or for manufacture of a drug for preventing and/or treating a PD-L1-related disease. The PD-L1-related disease comprises an inflammatory disease, autoimmune disease, etc., including but not limited to a breast cancer, liver cancer, gastric cancer, colorectal cancer, leukemia, lung cancer, kidney tumors, small intestine cancer, prostate cancer, colorectal cancer, prostate cancer, cervical cancer, lymphoma, bone cancer, adrenal tumors, or bladder tumors.

It should be understood that a cancer that does not respond to the treatment of one or more checkpoint inhibitors (e.g., an antibody that binds to PD-L1, CTLA-4, or CD47, etc.), such as a pancreatic cancer or prostate cancer, is referred to as a cold tumor. Regarding a cancer that responds to treatment with one or more checkpoint inhibitors, such as an antibody that binds to PD-L1, CTLA-4, or CD47, etc., such tumors are also referred to as a warm or hot tumor. Such tumors are considered to have higher levels of tumor infiltrating lymphocytes (TILs) and/or higher tumor mutation loads compared to tumors that do not respond to checkpoint inhibitor treatment.

The present invention provides a preferred antibody-drug conjugate, which has excellent anti-tumor activity and also exhibits extremely significant anti-tumor activity and response rate in cold tumors and tumor models with low PD-L1 expression.

### Pharmaceutical Composition

The present invention further provides a composition. Preferably, the composition is a pharmaceutical composition, which contains the antibody described as above or an active fragment or fusion protein thereof, and a pharmaceutically acceptable carrier or excipient, and optionally other biologically active substances. Generally, these substances may be formulated in a non-toxic, inert and pharmaceutically acceptable aqueous carrier medium, wherein the pH is generally about 5-8, preferably about 6-8, although the pH value may vary depending on the nature of the formulated substance and the condition to be treated. The formulated pharmaceutical composition may be administered by conventional routes, including (but not limited to): intraperitoneal, intravenous, or topical administration.

The pharmaceutical composition of the present invention contains a safe and effective amount (such as 0.001-99 wt %, preferably 0.01-90 wt %, more preferably 0.1-80 wt %) of the antibody of the present invention as described above (or a conjugate thereof) and a pharmaceutically acceptable carrier or excipient. Such carriers include (but are not limited to): saline, buffer, glucose, water, glycerol, ethanol, and a combination thereof. The pharmaceutical preparation should match the mode of administration. The pharmaceutical composition of the present invention may be prepared in the form of an injection, for example, by conventional methods using physiological saline or an aqueous solution containing glucose and other adjuvants. Pharmaceutical compositions such as injections and solutions are preferably manufactured under sterile conditions. The dosage of the active ingredient is a therapeutically effective amount, for example, about 10 μg/kg body weight to about 50 mg/kg body weight per day. In addition, the polypeptide of the present invention may also be used with other therapeutic agents.

When using a pharmaceutical composition, a safe and effective amount of the immunoconjugate is administered to a mammal, wherein the safe and effective amount is usually at least about 10 μg/kg body weight, and in most cases does not exceed about 50 mg/kg body weight, preferably the dosage is about 10 μg/kg body weight to about 10 mg/kg body weight. Of course, factors such as the route of administration and the patient's health condition should also be considered for specific dosages, which are all within the skill range of skilled physicians.

### The Main Advantages of the Present Invention Include

(a) The anti-human PD-L1 nanobody of the present invention can be highly specific to human PD-L1 protein with the correct spatial structure, has strong affinity, and is easy to produce.
(b) The present invention provides a combined treatment regimen of PD-L1 nanobodies and TLR7 agonists, and demonstrates significant in vivo anti-tumor activity, indicating that PD-L1 antibody therapy and TLR7 immune agonists are rational for combination and have synergistic antitumor effect.
(c) The present invention developed a dual-targeting nanobody drug conjugate targeting human PD-L1 and TLR7, which may promote the upregulation of PD-L1 expression in tumor cells and coordinate the innate immunity and adaptive anti-tumor immune response in the tumor, so that it may show excellent tumor growth inhibition in a variety of tumors such as “cold” tumors and tumors with low PD-L1 expression, in which the PD-L1 antibody treatment effect is poor.
(d) The PD-L1 and TLR7 dual-targeting nanobody-drug conjugate of the present invention may target the tumor immune microenvironment and reconstitute the tumor immune microenvironment, thereby increasing the infiltration of anti-tumor immune cells and reducing the infiltration of immunosuppressive cells.

The present invention is further illustrated by the following specific examples. It should be understood that these examples are only for illustrating the present invention and not intend to limit the scope of the present invention. The conditions of the experimental methods not specifically indicated in the following examples are usually in accordance with conventional conditions as described in Sambrook and Russell et al., Molecular Cloning: A Laboratory Manual (third edition) (2001) (CSHL press), or according to the conditions recommended by the manufacturer. Unless otherwise stated, percentages and parts are calculated by weight.

### Example 1: Screening Nanobodies Against Human PD-L1

### 1.1 Human PD-L1 Protein Expression

The human PD-L1 (ECD)-pFUSE-hIgG1-Fc vector was constructed and subjected to plasmid amplification. The human PD-L1 protein was expressed in mammalian cells HEK293F and purified using a Protein A affinity column.

### 1.2 Immunization of Animals and Library Construction

Camels were immunized with the antigen protein obtained in step 1.1. After 5 immunizations, peripheral blood was drawn, and peripheral blood mononuclear cells were separated. cDNAs were obtained by RNA extraction and reverse transcription. The VHH gene fragment was obtained by nested PCR amplification and linked to the phage plasmid vector. The ligation product was transferred into Escherichia coli electrocompetent TG1 via electroporation to complete the construction of the nanobody library against human PD-L1 protein.

### 1.3 Library Panning

Auxiliary phages were added to the 10-fold storage capacity of the nanobody library to obtain nanobody display phages. The 96-well ELISA plate was coated with 5 μg/mL NeutrAvidin solution (100 μL per well) at 4° C. overnight. The next day, it was blocked with 2% skim milk powder at room temperature for 2 h and washed 5 times with 20 mM HEPES (pH7.5) and 150 mM NaCl solution. The control group and the experimental group were set up. 100 μL of 200 nM PD-L1-biotin protein dilution was added, shaken at room temperature (700 rpm), incubated for 15 min, and washed 5 times with 20 mM HEPES (pH7.5) and 150 mM NaCl solution. Then 100 μL of phage dilution (1×10¹³ cfu/mL) was added. The solution was shaken at room temperature (700 rpm) and incubated for 2 h, and washed 5 times with 20 mM HEPES (pH7.5), 150 mM NaCl solution to remove unbound phages. Then 100 μL of 0.25 mg/mL trypsin was added and shaken at room temperature (700 rpm) for 30 min to dissociate the specifically bound phages. The inhibitor was added to terminate digestion. TG1 cells were infected with the eluted phages for the second round of panning. The above operation was repeated for 2-3 times until the positive clones were enriched.

### 1.4 ELISA Identification of Positive Clones

After several rounds of panning, TG1 competent cells in the logarithmic phase of growth were infected with the eluted phages, gradiently diluted and spread on the plate for overnight culture. 96 clones were picked and inoculated into a 96-well round-bottom plate with 100 μL of culture medium per well and left to be incubated overnight as the master plate. Then 10 μL of overnight cultured bacterial solution was pipetted into a 96-well deep-bottom plate with 1 mL of culture medium per well to induce nanobody expression which was then subjected to crude purification. The 96-well ELISA plate was coated with 5 μg/mL NeutrAvidin solution at 4° C., 700 rpm, overnight. The next day, 2% skim milk powder was added to block the plate at room temperature and washed 3 times with a solution containing bovine serum albumin BSA. 100 μL 3 μg/mL PD-L1 protein was added and incubated at room temperature for 30 minutes, then washed. The crudely extracted nanobody was added and incubated at room temperature for 1 h followed by washing. Mouse anti-HA primary antibody was added and the mixture was incubated at room temperature for 1 hour followed by washing. Goat anti-mouse alkaline phosphatase-labeled secondary antibody was added and the mixture was incubated at room temperature for 1 hour followed by washing. Alkaline phosphatase colorimetric solution was added for 10 minutes, and the absorption value was detected at 405 nm on a microplate reader. A well with an absorption value that is more than three times the absorption value of the control group is preliminarily determined as a positive well. The positive clones were transferred to a shaking tube for culture in order to extract the plasmid and then subjected to sequencing. Finally, 11 candidate positive clones were obtained.

### Example 2: Expression and Identification of Anti-Human PD-L1 Nanobodies

After preparation of the linearized pFUSE-mIgG2b-Fc and pFUSE-hIgG1-Fc vectors, the candidate nanobody sequences were homologously recombined into the pFUSE-mIgG2b-Fc and pFUSE-hIgG1-Fc vectors, and then the candidate nanobodies were expressed using mammalian cells HEK293F.

The 11 preferred anti-human PD-L1 nanobodies obtained in the present invention are named h_Nb1, h_Nb2, h_Nb4, h_Nb5, h_Nb6, h_Nb9, h_Nb12, h_Nb13, h_Nb19, h_Nb26, and h_Nb30, respectively.

Wherein, the VHH sequences of h_Nb1, h_Nb2, h_Nb4, h_Nb5, h_Nb6, h_Nb9, h_Nb12, h_Nb13, h_Nb19, h_Nb26, and h_Nb30 are shown in SEQ ID NOs.: 1, 5, 9, 13, 17, 20, 24, 28, 32, 36, and 39, respectively, and the CDRs are shown in Table 1.

TABLE 1
VHH and CDR sequences of anti-human PD-L1 antibodies

| SEQ | | |
|---|---|---|
| ID | | |
| NO. | Name | Sequence |
| 1 | h_Nb1 VHH | DVQLQESGGGLVQPGGSLRLSCAASGFTFSSYAMSWVRQAPGK |
| | | GLEWVSDINSGGDNTDYADSVKGRFTISRDNAKNTLYLQMNSL |
| | | KPEDTAVYYCARGPKLCFSSWGQGTQVTVSS |
| 2 | h_Nb1 CDR1 | GFTFSSYA |
| 3 | h_Nb1 CDR2 | INSGGDNT |
| 4 | h_Nb1 CDR3 | ARGPKLCFSS |
| 5 | h_Nb2 VHH | DVQLQESGGGLVQPGGSLRLSCAASGFTFGDRWMYWLRQAPG |
| | | KGLEWVSSINPAGRSTYYADSVKGRFTISRDNAKNTLYLQMNS |
| | | LKSEDTAVYYCTKDPGGYQKGQGTQVTVSS |
| 6 | h_Nb2 CDR1 | GFTFGDRW |
| 7 | h_Nb2 CDR2 | INPAGRST |
| 8 | h_Nb2 CDR3 | TKDPGGYQ |
| 9 | h_Nb4 VHH | DVQLQESGGGLVQAGGSLRLSCAAPSRMFSGYTMGWYRQAPG |
| | | KQRELVAQIANGGFTDYIDSVKGRFSISRDTVLNMMYLQMNSL |
| | | KPEDTAVYYCNLVLVNGIPTRNWGQGTQVTVSS |
| 10 | h_Nb4 CDR1 | SRMFSGYT |
| 11 | h_Nb4 CDR2 | IANGGFT |
| 12 | h_Nb4 CDR3 | NLVLVNGIPTRN |
| 13 | h_Nb5 VHH | DVQLQESGGGAVQSGESLRLSCAASGRDLMFYDVAWFRQAPG |
| | | KEREGVSYIRSSGTLTKYADSVKGRFTISRDNDKNTVSLQMNSL |
| | | NPEDTAKYYCAARLQSATSHSDLREDEFNSWGQGTQVTVSS |
| 14 | h_Nb5 CDR1 | GRDLMFYD |
| 15 | h_Nb5 CDR2 | IRSSGTLT |
| 16 | h_Nb5 CDR3 | AARLQSATSHSDLREDEFNS |
| 17 | h_Nb6 VHH | DVQLQESGGGLAQPGGSLRLSCAASGFTFSSYAMSWARQAPGK |
| | | GLEWVAGIYRGGSDTYYADSVKGRFTISRDNTKNTLYLQMNSL |
| | | KPEDTALYYCIQGWGLGYANYDYWGQGTQVTVSS |
| 2 | h_Nb6 CDR1 | GFTFSSYA |
| 18 | h_Nb6 CDR2 | IYRGGSDT |
| 19 | h_Nb6 CDR3 | IQGWGLGYANYDY |
| 20 | h_Nb9 VHH | DVQLQESGGGLVQPGGSLRLSCASSGSIFGLGVKGWYRQPPGN |
| | | QREMVAQIISGGRISYADSVKGRFTISRDNAKNTVYLQMNSLKP |
| | | EDTAVYYCNAWGPGQRNYWGRGTQVTVSS |
| 21 | h_Nb9 CDR1 | GSIFGLGV |
| 22 | h_Nb9 CDR2 | IISGGRI |
| 23 | h_Nb9 CDR3 | NAWGPGQRNY |
| 24 | h_Nb12 VHH | DVQLQESGGGLVQAGGSLRLSCAASGRTFSSRAMGWFRQAPG |
| | | KEREFVAAISGSGRNTYYADSVKGRFTISRDNAKNSVNLQMNS |
| | | LKPEDTAVYYCNAAGGGITIATMTQRTQYDYWGQGTQVTVSS |
| 25 | h_Nb12CDR1 | GRTFSSRA |
| 26 | h_Nb12 CDR2 | ISGSGRNT |
| 27 | h_Nb12 CDR3 | NAAGGGITIATMTQRTQYDY |
| 28 | h_Nb13 VHH | DVQLQESGGGLVQPGGSLRLSCAASGSIFSINAMGWYRQAPGK |
| | | SRELVAAITSGGSTNYADSVKGRFTISRDTAKNTVYLLMNSLEP |
| | | EDTAVYYCNVERRRPPHYLSEYDFDWGQGTQVTVSS |
| 29 | h_Nb13CDR1 | GSIFSINA |
| 30 | h_Nb13 CDR2 | ITSGGST |
| 31 | h_Nb13 CDR3 | NVERRRPPHYLSEYDFD |
| 32 | h_Nb19 VHH | DVQLQESGGGLVQAGGSLRLSCAASGRTFSGYAMAWFRQAPG |
| | | KEREFVAAISGSGGRTYYADSRFTISRDNAKNTLYLQMNSLKPE |
| | | DTAGYYCAAAGEGITIATMTQRTQYDYWGQGTQVTVSS |
| 33 | h_Nb19CDR1 | GRTFSGYA |
| 34 | h_Nb19 CDR2 | ISGSGGRT |
| 35 | h_Nb19 CDR3 | AAAGEGITIATMTQRTQYDY |
| 36 | h_Nb26 VHH | DVQLQESGGGLVQPGGSLRLSCAAPGSIFRINAMGWYRQAPGK |
| | | QRELVAAITSGGSTNYADSVQGRFTISRDNAKNTVYLQMNSLK |
| | | PEDTAVYYCNVERRRPPHYYSDYDFDWGQGTQVTVSS |
| 37 | h_Nb26CDR1 | GSIFRINA |
| 30 | h_Nb26 CDR2 | ITSGGST |
| 38 | h_Nb26 CDR3 | NVERRRPPHYYSDYDFD |
| 39 | h_Nb30 VHH | DVQLQESGGGLVQPGESLRVSCAASGFTFSNYYMTWVRQAPG |
| | | KGLEWVSFLNPSSSERFYADSVKGRFTISRDNAKNTLDLLMHSL |
| | | KPEDTALYYCARHLKYNWAPNYDYWGQGTQVTVSS |
| 40 | h_Nb30 CDR1 | GFTFSNYY |
| 41 | h_Nb30 CDR2 | LNPSSSER |
| 42 | h_Nb30 CDR3 | ARHLKYNWAPNYDY |
| 98 | h_Nb1_C103K | ARGPKLKFSS |
| | CDR3 | |
| 99 | h_Nb1_C103M | ARGPKLMFSS |
| | CDR3 | |
| 100 | h_Nb1_C103W | ARGPKLWFSS |
| | CDR3 | |
| 101 | h_Nb1_G99R | ARRPKLCFSS |
| | CDR3 | |
| 102 | h_Nb1_G99Q | ARQPKLCFSS |
| | CDR3 | |
| 103 | h_Nb1_G99W | ARWPKLCFSS |
| | CDR3 | |
| 104 | h_Nb1_G99Y | ARYPKLCFSS |
| | CDR3 | |
| 105 | h_Nb2_G30R | GFTFRDRW |
| | CDR1 | |
| 106 | h_Nb2_G30F | GFTFFDRW |
| | CDR1 | |
| 107 | h_Nb2_G30W | GFTFWDRW |
| | CDR1 | |
| 108 | h_Nb2_G101R | TKDPRGYQ |
| | CDR3 | |
| 109 | h_Nb2_G101W | TKDPWGYQ |
| | CDR3 | |
| 110 | h_Nb2_G101Y | TKDPYGYQ |
| | CDR3 | |
| 111 | h_Nb2_F29R | GFTRGDRW |
| | CDR1 | |
| 112 | h_Nb2_F29W | GFTWGDRW |
| | CDR1 | |
| 113 | h_Nb2_P100R | TKDRGGYQ |
| | CDR3 | |
| 114 | h_Nb2_P100Q | TKDQGGYQ |
| | CDR3 | |
| 115 | h_Nb2_P100E | TKDEGGYQ |
| | CDR3 | |

### Example 3: Preliminary Evaluation of In Vitro Activity of Anti-Human PD-L1 Nanobodies

### 3.1 Determination of Human PD-L1 Binding Activity of Candidate Anti-Human PD-L1 Nanobodies

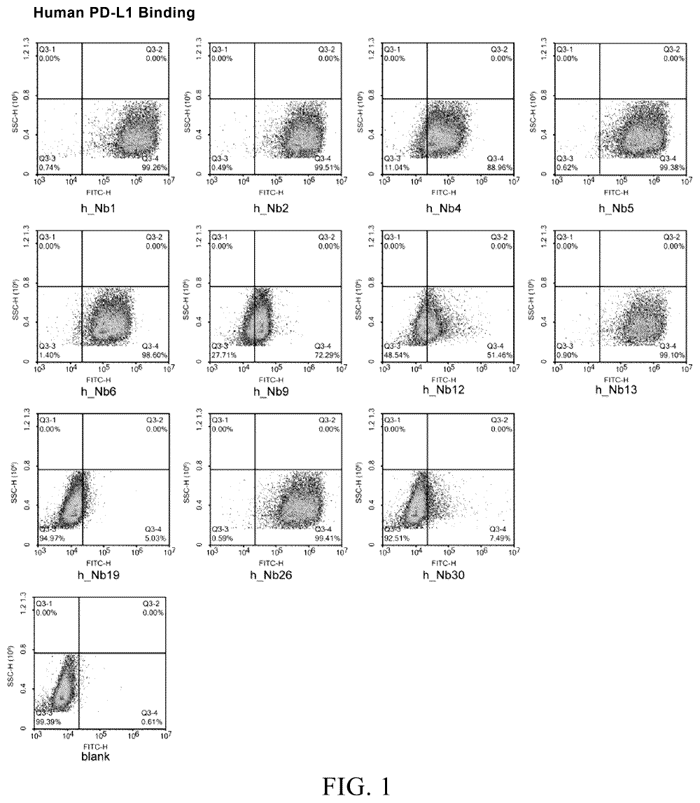

The stably transfected cell line HEK293T/hPD-L1 was digested and centrifuged, and the supernatant was removed. The cells were washed once with PBS and the cell density was adjusted to 2.5×10⁶ cells/mL. 100 μL of cell suspension was added to each cell sample, i.e., 2.5×10⁵ cells/sample. Different anti-human PD-L1 candidate nanobodies (at a concentration of 10 μg/ml) were added into the samples. The samples were incubated in a 4° C. refrigerator for 20 min and then centrifuged to remove the supernatant, and washed once with PBS. The diluted antibody anti-human IgG Fc (FITC) was used as a secondary antibody to resuspend the above cells. The abtained samples were incubated in a 4° C. refrigerator for 20 min and centrifuged to remove the supernatant. The cells were washed twice with PBS, transferred to a flow tube; and detected on a flow cytometer to obtain the binding activity of the candidate nanobody with human PD-L1, as shown in FIG. 1.

The experimental results suggest that a total of 9 candidate nanobodies, including h_Nb1, h_Nb2, h_Nb4, h_Nb5, h_Nb6, h_Nb9, h_Nb12, h_Nb13, and h_Nb26, all maintain good binding activity to human PD-L1.

### 3.2 Determination of Activity of Candidate Anti-Human PD-L1 Nanobodies in Blocking Human PD-1/PD-L1

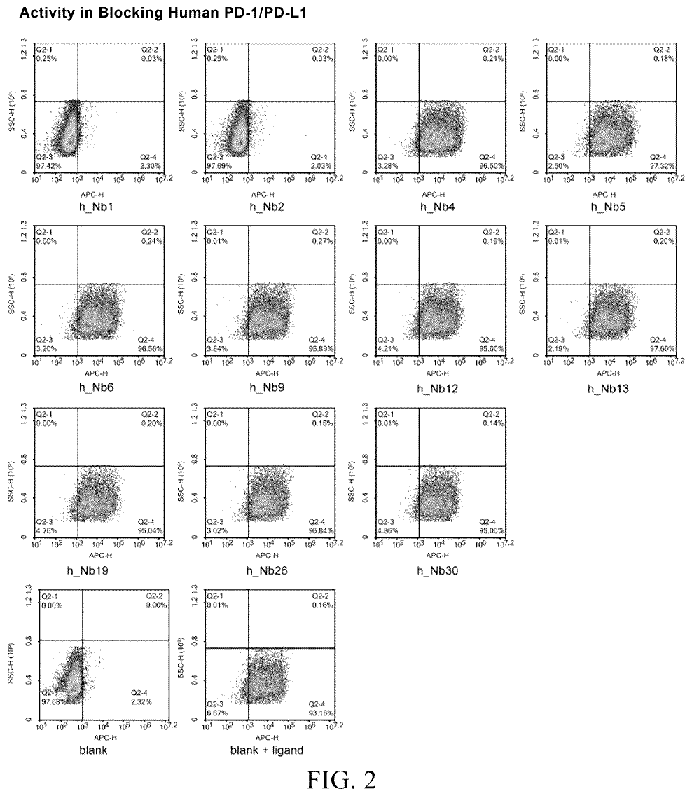

The stably transfected cell line HEK293T/hPD-L1 was digested and centrifuged to remove the supernatant. The cells were washed once with PBS and the cell density was adjusted to 2.5×10⁶ cells/mL. 100 μL of cell suspension was added to each cell sample, i.e., 2.5×10⁵ cells/sample. The human PD-1-his protein (at a concentration of 1 μg/mL) and candidate anti-human PD-L1 nanobodies (at a concentration of 10 μg/ml) were added to the samples respectively. The abtained samples were incubated in a refrigerator at 4° C. for 20 min and centrifuged to remove the supernatant. The obtained samples were washed once with PBS. The diluted antibody anti-his-APC was used as the secondary antibody to resuspend the above cells. The cells were incubated in a refrigerator at 4° C. for 20 min and centrifuged to remove the supernatant. The cells were washed twice with PBS, transferred to a flow tube; and detected by flow cytometry. The PD-1/PD-L1 blocking activity of the candidate nanobody was obtained, as shown in FIG. 2.

The experimental results suggest that the candidate nanobodies h_Nb1 and h_Nb2 can effectively block the binding activity of PD-1/PD-L1 and have significant blocking effects.

### Example 4: Evaluation of In Vitro Activity of Anti-Human PD-L1 Nanobodies

### 4.1 Determination of IC₅₀ Activity of Candidate Anti-Human PD-L1 Nanobodies in Blocking Human PD-1/PD-L1 Binding

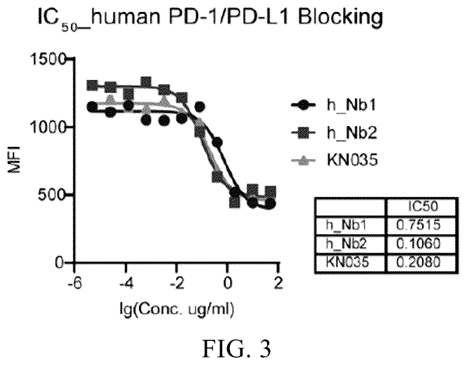

The cell samples were prepared by digesting the stably transfected cell line HEK293T/hPD-L1. Different concentrations of nanobodies h_Nb1 or h_Nb2 or positive control antibody KN035 (sequence from the IMGT database) and human PD-1-his protein (concentration of 1 μg/mL) were added to the samples. After incubation and centrifugation, anti-his-APC was used as the secondary antibody for incubation and staining; and then the flow cytometer was used for detection to obtain the IC₅₀ value of the candidate nanobody in blocking the binding of human PD-1/PD-L1, as shown in FIG. 3.

The experimental results suggest that the IC₅₀ activities of the candidate nanobodies h_Nb1 and h_Nb2 were comparable to those of the positive control antibody KN035, and that the in vitro activity of h_Nb2 was even superior to that of KNO35.

### Example 5: Preparation of TLR7 Agonist SZU-101

The molecular structure of the TLR7 small molecule agonist used to link the antibody is as follows:

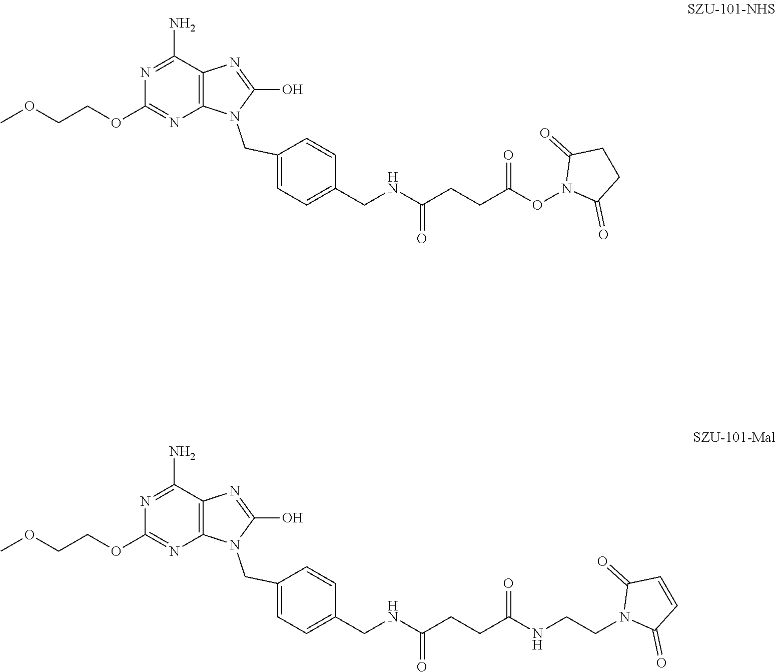 [SMILES: COCCOc1nc(N)c2nc(O)n(Cc3ccc(CNC(=O)CCC(=O)ON4C(=O)CCC4=O)cc3)c2n1 ; COCCOc1nc(N)c2nc(O)n(Cc3ccc(CNC(=O)CCC(=O)NCCN4C(=O)C=CC4=O)cc3)c2n1]

The synthesis of SZU-101 and the SZU-101 derivatives used for coupling as described above may be carried out by the following method or a similar method.

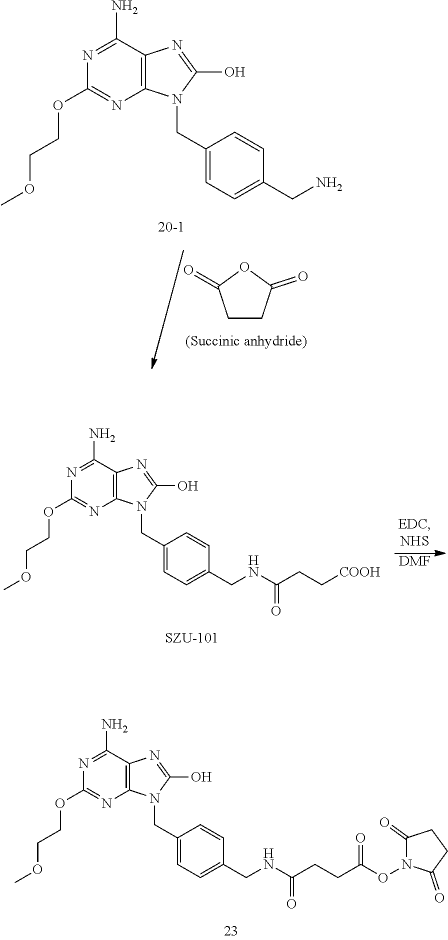

Compound 20-1 was dissolved in anhydrous DMSO, cooled at 10° C., and an equivalent amount of succinic anhydride was added, and the mixture was stirred at natural room temperature for 24 hours. The mixed reactants were poured into 20 times the volume of water, and a large amount of white solid compound SZU-101 was precipitated.

SZU-101 (1 eq), NHS (1.2 eq) and EDC (1.3 eq) were dissolved in anhydrous DMF and stirred at room temperature for 4 h. The reaction was quenched and the reaction solution was poured into dichloromethane and filtered to dryness to obtain compound 23, namely SZU-101-NHS, as a white solid.

SZU-101-Mal may be prepared in a similar manner.

### Example 6: Preparation of PD-L1 and TLR7 Dual-Targeting Nanobody-Drug Conjugates

SZU-101, EDCI and NHS were dissolved in DMSO and stirred at room temperature for 3 h. The reaction progress was monitored by LC-MS. After the reaction was complete, ten times the amount of double distilled water was added. The product was filtered and vacuum-dried to obtain SZU-101-NHS active ester.

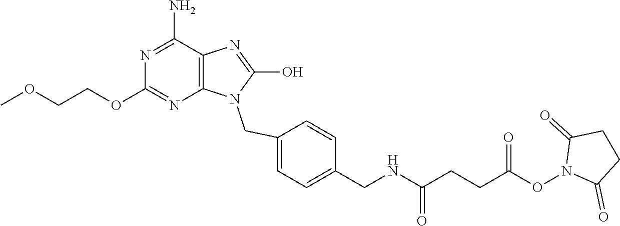 [SMILES: COCCOc1nc(N)c2nc(O)n(Cc3ccc(CNC(=O)CCC(=O)ON4C(=O)CCC4=O)cc3)c2n1]

Subsequently, the active ester was dissolved in DMSO. The antibody and the small molecule were reacted at a molar ratio of 1:10. A certain amount of small molecule active ester was added to the candidate antibody, and stirred at 4° C. for 4 hours. After the reaction was complete, PBS was added into the mixture and stirred. The small molecules were filtered through a 10 kD biological filter membrane to obtain novel dual-targeting nanobody conjugated compounds h_Nb1-SZU-101 and h_Nb2-SZU-101. For the conjugated compounds obtained by the reaction, the antibodies were first denatured to open the disulfide bonds, and then the samples were identified by using XevoG2XSQTOF mass spectrometer, and the degree of conjugation was calculated mainly based on the increased molecular weight of the newly conjugated compounds compared with the unconjugated antibodies.

### Example 7: Determination of In Vitro Activity of PD-L1 and TLR7 Dual-Targeting Nanobody-Drug Conjugates

### 7.1 Determination of PD-L1 Binding Activity of PD-L1 and TLR7 Dual-Targeting Nanobody Drug Conjugates

The stable cell line HEK293T/hPD-L1 was digested and centrifuged to remove the supernatant. The cells were washed once with PBS and the cell density was adjusted to 2.5×10⁶ cells/mL. 100 μL of cell suspension was added to each cell sample, i.e., 2.5×10⁵ cells/sample. Different concentrations of coupling compounds h_Nb1-SZU-101 or h_Nb2-SZU-101 and their corresponding naked antibodies were added to the samples. The abtained samples were incubated in a refrigerator at 4° C. for 20 min and centrifuged to remove the supernatant. The cells were washed once with PBS. The diluted antibody anti-human IgG Fc (FITC) was used as the secondary antibody to resuspend the above cells. The abtained samples were incubated in a refrigerator at 4° C. for 20 min and centrifuged to remove the supernatant. The cells were washed twice with PBS, transferred to a flow tube; and detected on a flow cytometer to obtain the EC₅₀ values of the nanobodies before and after coupling.

### 7.2 Determination of Activity of PD-L1 and TLR7 Dual-Targeting Nanobody-Drug Conjugates in Blocking PD-1/PD-L1

The stably transfected cell line HEK293T/hPD-L1 was digested and centrifuged to remove the supernatant. The cells were washed once with PBS and the cell density was adjusted to 2.5×10⁶ cells/mL. 100 μL of cell suspension was added to each cell sample, i.e., 2.5×10⁵ cells/sample. The human PD-1-his protein (at a concentration of 1 μg/mL) and different concentrations of h_Nb1-SZU-101 or h_Nb2-SZU-101 and their corresponding naked antibodies were added to the samples respectively. The abtained samples were incubated in a refrigerator at 4° C. for 20 min, centrifuged to remove the supernatant, and washed once with PBS. The diluted antibody anti-his-APC was used as the secondary antibody to resuspend the above cells. The abtained samples were incubated in a refrigerator at 4° C. for 20 min and centrifuged to remove the supernatant. The cells were washed twice with PBS, transferred to a flow tube; and detected on a flow cytometer to obtain the IC₅₀ values of the nanobodies before and after coupling.

### Example 8: Evaluation of In Vivo Anti-Tumor Activity of Anti-Human PD-L1 and TLR7 Dual-Targeting Nanobody-Drug Conjugates

As described in Example 6, anti-human PD-L1 and TLR7 dual-targeting nanobody drug conjugates h_Nb1-SZU-101 and h_Nb2-SZU-101 were prepared.

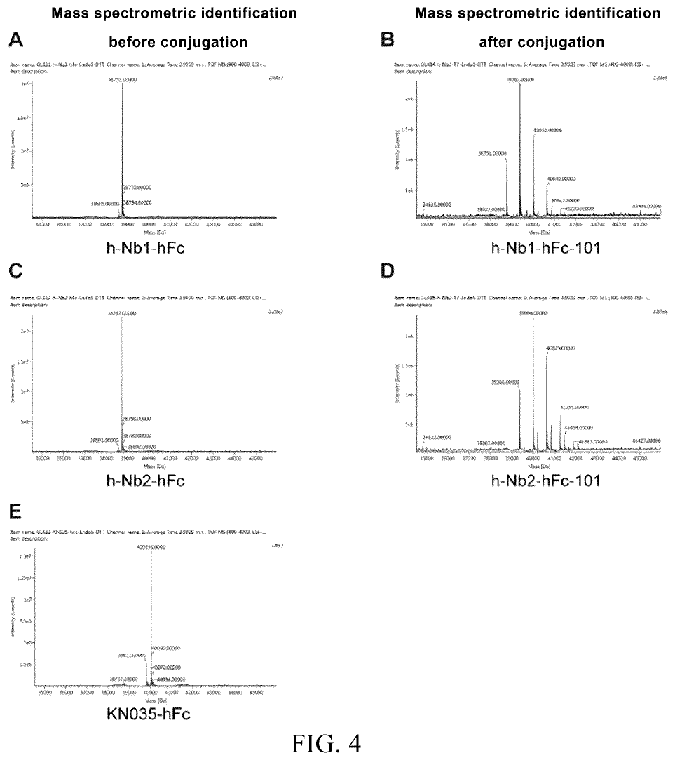

As shown in FIG. 4, the candidate nanobodies h_Nb1, h_Nb2 and the positive control antibody KN035 all have high purity. The coupling degree of the nanobody conjugate h_Nb1-SZU-101 identified by mass spectrometry was about 3, and the coupling degree of h_Nb2-SZU-101 was about 5.

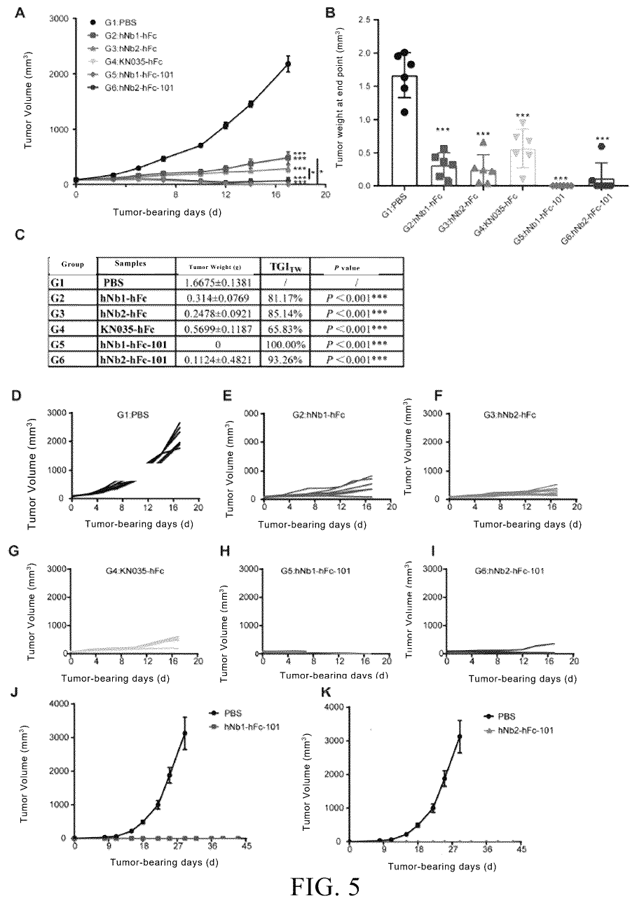

To further evaluate the in vivo anti-tumor activity of anti-human PD-L1 and TLR7 dual-targeting nanobody conjugates, the CT26/hPD-L1 tumor model established by using BALB/c mice with dual humanization of PD-1/PD-L1 targets was used. When the average tumor volume reached 80-100 mm³, the mice were randomly divided into 6 groups according to the tumor volume, with 6 mice in each group; the day of grouping was defined as DO, and the drugs were administered on DO, D3, D7, D10, D13, and D16, for a total of 6 times, with a dose of 10 mg/kg by intraperitoneal administration. The tumor size was measured 2-3 times a week, and the tumor growth curve was plotted. The tumors that did not disappear were dissected on Day 17, and the tumor inhibition rate was calculated, as shown in FIGS. 5A-5I.

The experimental results showed that the tumor inhibition rates of the candidate nanobodies h_Nb1 (SEQ ID NO. 1), h_Nb2 (SEQ ID NO. 5) and the positive control antibody KN035 were 81.17%, 85.14%, and 65.83%, respectively. The inhibitory activities of h_Nb1 and h_Nb2 were better than that of the control antibody KN035. Unexpectedly, the anti-tumor activity of the nanobody-drug conjugates h_Nb1-SZU-101 and h_Nb2-SZU-101 was significantly better than those of h_Nb1 and h_Nb2, with tumor inhibition rates of 100.00% and 93.26%, respectively, which strongly inhibited the progression of tumors in vivo. In addition, h_Nb1-SZU-101 caused 100% tumor-bearing mice to experience tumor regression (FIG. 5 H), and h_Nb2-SZU-101 caused 66.7% tumor-bearing mice to experience tumor regression (FIG. 5I). The mice with tumor-regression were subjected to tumor-rebeared experiments 28 days after the last administration of the efficacy test. Six BALB/c-hPD1/hPDL1 mice that had not been tumor-beared were re-selected as the control group and were tumor-beared (i.e., CT26/hPD-L1 tumors) at the same time as the mice with tumor-regression to observe the tumor growth curve of the mice. The results showed that the mice with tumor-regression no longer had tumor growth within 45 days of tumor bearing (FIGS. 5J-5K), indicating that the conjugated compound induced an effective anti-tumor immune memory.

### Example 9: Humanization of Anti-Human PD-L1 Nanobodies

The CDR Grafting method was used to replace camel antibody FR with human antibody FR to reduce immunogenicity. Firstly, homology modeling is carried out on the candidate antibodies to identify key amino acid residue sites, and then the candidate nanobody sequences were used as templates to search for homology structures in the structure database, and the optimal structural sequences were selected for sequence replacement to finally obtain the humanized antibody sequences. At the same time, it is necessary to consider retaining the key sites in the framework region that potentially affect the function of CDRs.

In the present invention, 11 preferred anti-human PD-L1 nanobodies were humanized, wherein the corresponding sequences of the humanized antibodies are as follows:
humanized sequences of h_Nb1 are h_Nb1_1, h_Nb1_2, h_Nb1_3, h_Nb1_4, and h_Nb1_5, respectively;
humanized sequences of h_Nb2 are h_Nb2_1, h_Nb2_2, h_Nb2_3, h_Nb2_4, and h_Nb2_5, respectively;
humanized sequences of h_Nb4 are h_Nb4_1 and h_Nb4_2, respectively;
humanized sequences of h_Nb5 are h_Nb5_1, h_Nb5_2, and h_Nb5_3, respectively;
humanized sequences of h_Nb6 are h_Nb6_1 and h_Nb6_2, respectively;
humanized sequences of h_Nb9 are h_Nb9_1 and h_Nb9_2, respectively;
humanized sequences of h_Nb12 are h_Nb12_1 and h_Nb12_2, respectively;
humanized sequences of h_Nb13 are h_Nb13_1 and h_Nb13_2, respectively;
humanized sequences of h_Nb19 are h_Nb19_1 and h_Nb19_2, respectively;
humanized sequences of h_Nb26 are h_Nb26_1 and h_Nb26_2, respectively;
humanized sequences of h_Nb30 are h_Nb30_1 and h_Nb30_2, respectively.

The sequences of humanized antibodies are shown in Table 2.

TABLE 2
VHH sequences of humanized anti-human PD-L1 antibodies

| SEQ ID | | |
|---|---|---|
| NOs. | Names | Sequences |
| 43 | h_Nb1_1 | QVQLVESGGGLVQPGGSLRLSCAASGFTFSSYAMSWVRQAPGK |
| | | GLEWVSAINSGGDNTYYADSVKGRFTISRDNSKNTLYLQMNSL |
| | | RAEDTAVYYCARGPKLCFSSWGQGTLVTVSS |
| 44 | h_Nb1_2 | QVQLVESGGGLVQPGGSLRLSCAASGFTFSSYAMSWVRQAPGK |
| | | GLEWVSDINSGGDNTDYADSVKGRFTISRDNAKNTLYLQMNSL |
| | | RAEDTAVYYCARGPKLCFSSWGQGTQVTVSS |
| 45 | h_Nb1_3 | QVQLVESGGGLVQPGGSLRLSCAASGFTFSSYAMSWVRQAPGK |
| | | GLEWVSDINSGGDNTDYADSVKGRFTISRDNSKNTLYLQMNSL |
| | | RAEDTAVYYCARGPKLCFSSWGQGTQVTVSS |
| 46 | h_Nb1_4 | QVQLVESGGGLVQPGGSLRLSCAASGFTFSSYAMSWVRQAPGK |
| | | GLEWVSDINSGGDNTDYADSVKGRFTISRDNAKNTLYLQMNSL |
| | | KAEDTAVYYCARGPKLCFSSWGQGTQVTVSS |
| 47 | h_Nb1_5 | QVQLVESGGGLVQPGGSLRLSCAASGFTFSSYAMSWVRQAPGK |
| | | GLEWVSDINSGGDNTDYADSVKGRFTISRDNSKNTLYLQMNSL |
| | | KAEDTAVYYCARGPKLCFSSWGQGTQVTVSS |
| 48 | h_Nb2_1 | QVQLVESGGGLVQPGGSLRLSCAASGFTFGDRWMYWVRQAPG |
| | | KGLEWVSAINPAGRSTYYADSVKGRFTISRDNSKNTLYLQMNS |
| | | LRAEDTAVYYCTKDPGGYQWGQGTLVTVSS |
| 49 | h_Nb2_2 | QVQLLESGGGLVQPGGSLRLSCAASGFTFGDRWMYWLRQAPG |
| | | KGLEWVSSINPAGRSTYYADSVKGRFTISRDNSKNTLYLQMNSL |
| | | RAEDTAVYYCTKDPGGYQKGQGTQVTVSS |
| 50 | h_Nb2_3 | QVQLVESGGGLVQPGGSLRLSCAASGFTFGDRWMYWLRQAPG |
| | | KGLEWVSSINPAGRSTYYADSVKGRFTISRDNAKNSLYLQMNSL |
| | | RAEDTAVYYCTKDPGGYQKGQGTQVTVSS |
| 51 | h_Nb2_4 | QVQLLESGGGLVQPGGSLRLSCAASGFTFGDRWMYWVRQAPG |
| | | KGLEWVSSINPAGRSTYYADSVKGRFTISRDNSKNTLYLQMNSL |
| | | RAEDTAVYYCTKDPGGYQKGQGTQVTVSS |
| 52 | h_Nb2_5 | QVQLVESGGGLVQPGGSLRLSCAASGFTFGDRWMYWVRQAPG |
| | | KGLEWVSSINPAGRSTYYADSVKGRFTISRDNAKNSLYLQMNSL |
| | | RAEDTAVYYCTKDPGGYQKGQGTQVTVSS |
| 53 | h_Nb4_1 | QVQLVESGGGLVQPGGSLRLSCAASSRMFSGYTMGWVRQAPG |
| | | KGLEWVSAIANGGFTYYADSVKGRFTISRDNSKNTLYLQMNSL |
| | | RAEDTAVYYCNLVLVNGIPTRNWGQGTLVTVSS |
| 54 | h_Nb4_2 | QVQLLESGGGLVQPGGSLRLSCAASSRMFSGYTMGWYRQAPG |
| | | KGLEWVAQIANGGFTDYIDSVKGRFTISRDNSKNTMYLQMNSL |
| | | RAEDTAVYYCNLVLVNGIPTRNWGQGTQVTVSS |
| 55 | h_Nb5_1 | QVQLVESGGGLVQPGGSLRLSCAASGRDLMFYDVAWVRQAPG |
| | | KGLEWVSAIRSSGTLTYYADSVKGRFTISRDNSKNTLYLQMNSL |
| | | RAEDTAVYYCAARLQSATSHSDLREDEFNSWGQGTLVTVSS |
| 56 | h_Nb5_2 | QVQLQESGGGAVQSGESLRLSCAASGRDLMFYDVAWFRQAPG |
| | | KEREGVSYIRSSGTLTKYADSVKGRFTISRDNDKNTVSLQMNSL |
| | | NPEDTAKYYCAARLQSATSHSDLREDEFNSWGQGTQVTVSS |
| 57 | h_Nb5_3 | QVQLVESGGGLVQPGGSLRLSCAASGRDLMFYDVAWVRQAPG |
| | | KGLEWVSYIRSSGTLTKYADSVKGRFTISRDNSKNTVYLQMNSL |
| | | RAEDTAVYYCAARLQSATSHSDLREDEFNSWGQGTQVTVSS |
| 58 | h_Nb6_1 | QVQLVESGGGLVQPGGSLRLSCAASGFTFSSYAMSWVRQAPGK |
| | | GLEWVSAIYRGGSDTYYADSVKGRFTISRDNSKNTLYLQMNSL |
| | | RAEDTAVYYCIQGWGLGYANYDYWGQGTLVTVSS |
| 59 | h_Nb6_2 | QVQLLESGGGLVQPGGSLRLSCAASGFTFSSYAMSWARQAPGK |
| | | GLEWVAGIYRGGSDTYYADSVKGRFTISRDNSKNTLYLQMNSL |
| | | RAEDTAVYYCIQGWGLGYANYDYWGQGTQVTVSS |
| 60 | h_Nb9_1 | QVQLVESGGGLVQPGGSLRLSCAASGSIFGLGVKGWVRQAPGK |
| | | GLEWVSAIISGGRIYYADSVKGRFTISRDNSKNTLYLQMNSLRA |
| | | EDTAVYYCNAWGPGQRNYWGQGTLVTV |
| 61 | h_Nb9_2 | QVQLVESGGGLVQPGGSLRLSCASSGSIFGLGVKGWVRQAPGK |
| | | GLEWVAQIISGGRISYADSVKGRFTISRDNAKNTVYLQMNSLRA |
| | | EDTAVYYCNAWGPGQRNYWGRGTQVTVSS |
| 62 | h_Nb12_1 | QVQLVESGGGLVQPGGSLRLSCAASGRTFSSRAMGWVRQAPG |
| | | KGLEWVSAISGSGRNTYYADSVKGRFTISRDNSKNTLYLQMNS |
| | | LRAEDTAVYYCNAAGGGITIATMTQRTQYDYWGQGTLVTVSS |
| 63 | h_Nb12_2 | 2QVQLVESGGGLVQPGGSLRLSCAASGRTESSRAMGWFRQAPGK |
| | | ELEFVAAISGSGRNTYYADSVKGRFTISRDNAKNSVYLQMNSLR |
| | | AEDTAVYYCNAAGGGITIATMTQRTQYDYWGQGTQVTVSS |
| 64 | h_Nb13_1 | QVQLVESGGGLVQPGGSLRLSCAASGSIFSINAMGWVRQAPGK |
| | | GLEWVSAITSGGSTYYADSVKGRFTISRDNSKNTLYLQMNSLRA |
| | | EDTAVYYCNVERRRPPHYLSEYDFDWGQGTLVTVSS |
| 65 | h_Nb13_2 | EVQLVESGGGLVQPGGSLRLSCAASGSIFSINAMGWVRQAPGK |
| | | GLEWVAAITSGGSTNYADSVKGRFTISRDNAKNTVYLQMNSLR |
| | | AEDTAVYYCNVERRRPPHYLSEYDFDWGQGTQVTVSS |
| 66 | h_Nb19_1 | QVQLVESGGGLVQPGGSLRLSCAASGRTFSGYAMSWVRQAPG |
| | | KGLEWVSAISGSGGRTYYADSVKGRFTISRDNSKNTLYLQMNS |
| | | LRAEDTAVYYCAAAGEGITIATMTQRTQYDYWGQGTLVTVSS |
| 67 | h_Nb19_2 | 2QVQLVESGGGLVQPGGSLRLSCAASGRTFSGYAMAWFRQAPG |
| | | KGREFVAAISGSGGRTYYADSRFTISRDNAKNTLYLQMNSLRAE |
| | | DTAVYYCAAAGEGITIATMTQRTQYDYWGQGTQVTVSS |
| 68 | h_Nb26_1 | QVQLVESGGGLVQPGGSLRLSCAASGSIFRINAMGWVRQAPGK |
| | | GLEWVSAITSGGSTYYADSVKGRFTISRDNSKNTLYLQMNSLRA |
| | | EDTAVYYCNVERRRPPHYYSDYDFDWGQGTLVTVSS |
| 69 | h_Nb26_2 | QVQLVQSGAEVKKPGASVKVSCKASGSIFRINAMGWYRQAPGQ |
| | | GLEWVAAITSGGSTNYADSVQGRFTITRDNSTSTVYLELRSLRS |
| | | DDTAVYYCNVERRRPPHYYSDYDFDWGQGTQVTVSS |
| 70 | h_Nb30_1 | QVQLVESGGGLVQPGGSLRLSCAASGFTFSNYYMSWVRQAPGK |
| | | GLEWVSALNPSSSERYYADSVKGRFTISRDNSKNTLYLQMNSLR |
| | | AEDTAVYYCARHLKYNWAPNYDYWGQGTLVTVSS |
| 71 | h_Nb30_2 | EVQLVESGGGLVQPGGSLRLSCAASGFTFSNYYMTWVRQAPGK |
| | | GLEWVSFLNPSSSERFYADSVKGRFTISRDNAKNTLDLQMNSLR |
| | | AEDTAVYYCARHLKYNWAPNYDYWGQGTQVTVSS |

### Example 10: Affinity Maturation Optimization of CDRs of Nanobodies

The full-length amino acid sequences of preferred nanobodies that specifically bind to human PD-L1 in the above examples (SEQ ID No. 1 and SEQ ID No. 5) were annotated with the CCG method for the antibody CDR and FR regions. Suitable FR and CDR region templates were selected by antibody homology modeling to construct and select the optimal nanobody three-dimensional protein structure. The crystal structure of human PD-L1 protein was obtained from the PDB protein database. A preferred candidate library of PD-L1 nanobody-PD-L1 protein complex structures was obtained by protein complex homology modeling and protein-protein molecular docking methods. Based on the PD-1/PD-L1 in vitro competitive binding characteristics of the preferred antibodies, a suitable docking angle was selected to determine the optimal docking conformation. The key interaction sites of the selected nanobody-PD-L1 protein complex structure were analyzed, with attention paid to Van Der Waals forces, hydrogen bonds, ionic bonds, hydrophobic interactions, etc. The pairs of action sites with low affinity energy values between the nanobodies and the action sites on PD-L1 were focused on as affinity maturation point mutation objects. The nanobody-PD-L1 protein interacting residue pairs that met the above requirements were selected. The corresponding amino acid sites of the nanobody were subjected to point-mutation. Mutations to other 19 natural amino acids were tested respectively. The changes in the interaction force between the mutated sites and the corresponding sites on PD-L1 were analyzed, and the mutation mode that significantly improved the affinity was selected. The sites on the CDR region of the nanobodies that had weak interactions with the PD-L1 protein were all screened by the above point-mutation, and excellent mutation combinations were selected. The sequences generated by the candidate affinity maturation scheme screened at the computer level were obtained through gene synthesis, cloning, transfection and protein expression and purification. The affinity levels of the candidate antibodies and the original antibodies (SEQ ID NO. 1 and SEQ ID NO. 5) to PD-L1 and their activities in blocking PD-1/PD-L1 were compared by the ELISA method. Finally, the affinity maturation scheme of the CDR regions of the preferred antibodies was determined, and the sequences of the antibodies after affinity maturation were SEQ ID NO. 72 to SEQ ID NO. 97. Wherein, the original antibody corresponding to the sequences shown in SEQ ID NOs. 72-86 is h_Nb1 VHH (SEQ ID NO. 1); the original antibody corresponding to the sequences shown in SEQ ID NOs. 87-97 is h_Nb2 VHH (SEQ ID NO. 5).

TABLE 3
VHH sequences after affinity maturation of anti-human
PD-L1 antibodies

| | | | Improved |
|---|---|---|---|
| SEQ | | | value of |
| ID | | | relative |
| NO. | Names | Sequences | affinity |
| 72 | h_Nb1_ | DVQLQESGGGLVQPGGSLRLSCAASGFTFSSYAMSW | 5.1327 |
| | A61N | VRQAPGKGLEWVSDINSGGDNTDYNDSVKGRFTISR | |
| | | DNAKNTLYLQMNSLKPEDTAVYYCARGPKLCFSSWG | |
| | | QGTQVTVSS | |
| 73 | h_Nb1_ | DVQLQESGGGLVQPGGSLRLSCAASGFTFSSYAMSW | 4.6638 |
| | A61Q | VRQAPGKGLEWVSDINSGGDNTDYQDSVKGRFTISR | |
| | | DNAKNTLYLQMNSLKPEDTAVYYCARGPKLCFSSWG | |
| | | QGTQVTVSS | |
| 74 | h_Nb1_ | DVQLQESGGGLVQPGGSLRLSCAASGFTFSSYAMSW | 4.1233 |
| | A61K | VRQAPGKGLEWVSDINSGGDNTDYKDSVKGRFTISR | |
| | | DNAKNTLYLQMNSLKPEDTAVYYCARGPKLCFSSWG | |
| | | QGTQVTVSS | |
| 75 | h_Nb1_ | DVQLQESGGGLVQPGGSLRLSCAASGFTFSSYAMSW | 4.2832 |
| | D59R | VRQAPGKGLEWVSDINSGGDNTRYADSVKGRFTISR | |
| | | DNAKNTLYLQMNSLKPEDTAVYYCARGPKLCFSSWG | |
| | | QGTQVTVSS | |
| 76 | h_Nb1_ | DVQLQESGGGLVQPGGSLRLSCAASGFTFSSYAMSW | 4.9025 |
| | D59M | VRQAPGKGLEWVSDINSGGDNTMYADSVKGRFTISR | |
| | | DNAKNTLYLQMNSLKPEDTAVYYCARGPKLCFSSWG | |
| | | QGTQVTVSS | |
| 77 | h_Nb1_ | DVQLQESGGGLVQPGGSLRLSCAASGFTFSSYAMSW | 5.1213 |
| | D59F | VRQAPGKGLEWVSDINSGGDNTFYADSVKGRFTISRD | |
| | | NAKNTLYLQMNSLKPEDTAVYYCARGPKLCFSSWGQ | |
| | | GTQVTVSS | |
| 78 | h_Nb1_ | DVQLQESGGGLVQPGGSLRLSCAASGFTFSSYAMSW | 11.6979 |
| | D62W | VRQAPGKGLEWVSDINSGGDNTDYAWSVKGRFTISR | |
| | | DNAKNTLYLQMNSLKPEDTAVYYCARGPKLCFSSWG | |
| | | QGTQVTVSS | |
| 79 | h_Nb1_ | DVQLQESGGGLVQPGGSLRLSCAASGFTFSSYAMSW | 5.7977 |
| | D62Y | VRQAPGKGLEWVSDINSGGDNTDYAYSVKGRFTISR | |
| | | DNAKNTLYLQMNSLKPEDTAVYYCARGPKLCFSSWG | |
| | | QGTQVTVSS | |
| 80 | h_Nb1_ | DVQLQESGGGLVQPGGSLRLSCAASGFTFSSYAMSW | 3.3129 |
| | C103K | VRQAPGKGLEWVSDINSGGDNTDYADSVKGRFTISR | |
| | | DNAKNTLYLQMNSLKPEDTAVYYCARGPKLKFSSW | |
| | | GQGTQVTVSS | |
| 81 | h_Nb1_ | DVQLQESGGGLVQPGGSLRLSCAASGFTFSSYAMSW | 3.3768 |
| | C103M | VRQAPGKGLEWVSDINSGGDNTDYADSVKGRFTISR | |
| | | DNAKNTLYLQMNSLKPEDTAVYYCARGPKLMESSW | |
| | | GQGTQVTVSS | |
| 82 | h_Nb1_ | DVQLQESGGGLVQPGGSLRLSCAASGFTFSSYAMSW | 3.8328 |
| | C103W | VRQAPGKGLEWVSDINSGGDNTDYADSVKGRFTISR | |
| | | DNAKNTLYLQMNSLKPEDTAVYYCARGPKLWFSSW | |
| | | GQGTQVTVSS | |
| 83 | h_Nb1_ | DVQLQESGGGLVQPGGSLRLSCAASGFTFSSYAMSW | 12.1151 |
| | G99R | VRQAPGKGLEWVSDINSGGDNTDYADSVKGRFTISR | |
| | | DNAKNTLYLQMNSLKPEDTAVYYCARRPKLCFSSWG | |
| | | QGTQVTVSS | |
| 84 | h_Nb1_ | DVQLQESGGGLVQPGGSLRLSCAASGFTFSSYAMSW | 6.1589 |
| | G99Q | VRQAPGKGLEWVSDINSGGDNTDYADSVKGRFTISR | |
| | | DNAKNTLYLQMNSLKPEDTAVYYCARQPKLCFSSW | |
| | | GQGTQVTVSS | |
| 85 | h_Nb1_ | DVQLQESGGGLVQPGGSLRLSCAASGFTFSSYAMSW | 6.1572 |
| | G99W | VRQAPGKGLEWVSDINSGGDNTDYADSVKGRFTISR | |
| | | DNAKNTLYLQMNSLKPEDTAVYYCARWPKLCFSSW | |
| | | GQGTQVTVSS | |
| 86 | h_Nb1_ | DVQLQESGGGLVQPGGSLRLSCAASGFTFSSYAMSW | 7.0186 |
| | G99Y | VRQAPGKGLEWVSDINSGGDNTDYADSVKGRFTISR | |
| | | DNAKNTLYLQMNSLKPEDTAVYYCARYPKLCFSSWG | |
| | | QGTQVTVSS | |
| 87 | h_Nb2_ | DVQLQESGGGLVQPGGSLRLSCAASGFTFRDRWMY | 6.2276 |
| | G30R | WLRQAPGKGLEWVSSINPAGRSTYYADSVKGRFTISR | |
| | | DNAKNTLYLQMNSLKSEDTAVYYCTKDPGGYQKGQ | |
| | | GTQVTVSS | |
| 88 | h_Nb2_ | DVQLQESGGGLVQPGGSLRLSCAASGFTFFDRWMY | 5.1098 |
| | G30F | WLRQAPGKGLEWVSSINPAGRSTYYADSVKGRFTISR | |
| | | DNAKNTLYLQMNSLKSEDTAVYYCTKDPGGYQKGQ | |
| | | GTQVTVSS | |
| 89 | h_Nb2_ | DVQLQESGGGLVQPGGSLRLSCAASGFTFWDRWMY | 10.0189 |
| | G30W | WLRQAPGKGLEWVSSINPAGRSTYYADSVKGRFTISR | |
| | | DNAKNTLYLQMNSLKSEDTAVYYCTKDPGGYQKGQ | |
| | | GTQVTVSS | |
| 90 | h_Nb2_ | WLRQAPGKGLEWVSSINPAGRSTYYADSVKGRFTISR | 6.4982 |
| | G101R | DVQLQESGGGLVQPGGSLRLSCAASGFTFGDRWMY | |
| | | DNAKNTLYLQMNSLKSEDTAVYYCTKDPRGYQKGQ | |
| | | GTQVTVSS | |
| 91 | h_Nb2_ | WLRQAPGKGLEWVSSINPAGRSTYYADSVKGRFTISR | 5.9213 |
| | G101W | DVQLQESGGGLVQPGGSLRLSCAASGFTFGDRWMY | |
| | | DNAKNTLYLQMNSLKSEDTAVYYCTKDPWGYQKGQ | |
| | | GTQVTVSS | |
| 92 | h_Nb2_ | DVQLQESGGGLVQPGGSLRLSCAASGFTFGDRWMY | 8.4837 |
| | G101Y | WLRQAPGKGLEWVSSINPAGRSTYYADSVKGRFTISR | |
| | | DNAKNTLYLQMNSLKSEDTAVYYCTKDPYGYQKGQ | |
| | | GTQVTVSS | |
| 93 | h_Nb2_ | DVQLQESGGGLVQPGGSLRLSCAASGFTRGDRWMY | 1.4558 |
| | F29R | WLRQAPGKGLEWVSSINPAGRSTYYADSVKGRFTISR | |
| | | DNAKNTLYLQMNSLKSEDTAVYYCTKDPGGYQKGQ | |
| | | GTQVTVSS | |
| 94 | h_Nb2_ | DVQLQESGGGLVQPGGSLRLSCAASGFTWGDRWMY | 1.4356 |
| | F29W | WLRQAPGKGLEWVSSINPAGRSTYYADSVKGRFTISR | |
| | | DNAKNTLYLQMNSLKSEDTAVYYCTKDPGGYQKGQ | |
| | | GTQVTVSS | |
| 95 | h_Nb2_ | DVQLQESGGGLVQPGGSLRLSCAASGFTFGDRWMY | 7.2081 |
| | P100R | WLRQAPGKGLEWVSSINPAGRSTYYADSVKGRFTISR | |
| | | DNAKNTLYLQMNSLKSEDTAVYYCTKDRGGYQKGQ | |
| | | GTQVTVSS | |
| 96 | h_Nb2_ | DVQLQESGGGLVQPGGSLRLSCAASGFTFGDRWMY | 3.4102 |
| | P100Q | WLRQAPGKGLEWVSSINPAGRSTYYADSVKGRFTISR | |
| | | DNAKNTLYLQMNSLKSEDTAVYYCTKDQGGYQKGQ | |
| | | GTQVTVSS | |
| 97 | h_Nb2_ | DVQLQESGGGLVQPGGSLRLSCAASGFTFGDRWMY | 3.9779 |
| | P100E | WLRQAPGKGLEWVSSINPAGRSTYYADSVKGRFTISR | |
| | | DNAKNTLYLQMNSLKSEDTAVYYCTKDEGGYQKGQ | |
| | | GTQVTVSS | |

### Discussion

A scientifically reasonable, safe and effective combination therapy can also guide the design of new drug molecules. In the present invention, an anti-human PD-L1 and TLR7 dual-targeting nanobody drug conjugate was developed, which can coordinate innate and adaptive immune responses, target and reconstitute the tumor immune microenvironment, and increase the PD-L1 expression level in tumor tissues. It also exhibits extremely significant anti-tumor activities and response rate in “cold” tumors and tumor models with a low PD-L1 expression, demonstrating its clinical application value.

In the present invention, an anti-human PD-L1 and TLR7 dual-targeting nanobody drug conjugate was provided, which may be further developed into a tumor immunotherapy drug, and is particularly suitable for tumors with a low immunogenicity and/or a low PD-L1 expression.

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. An antibody-drug conjugate or a pharmaceutically acceptable salt thereof, wherein the structure of the antibody-drug conjugate is as shown in formula I:
$$Ab - {( J - U )}_{n} \qquad ( I )$$
wherein,
Ab is a PD-L1 antibody;
each U is independently a TLR agonist;
J is a chemical bond or linker;
n is 0 or a positive integer; and
“—” is a chemical bond, a connector or a linker;
wherein the PD-L1 antibody is a PD-L1 nanobody or a derivative antibody thereof, preferably a nanobody targeting human PD-L1 or a derivative antibody thereof; and the complementary determining region CDR of the VHH chain in the nanobody is selected from the group consisting of:
(1) CDR1 shown in SEQ ID NO: 2, CDR2 shown in SEQ ID NO: 3, and CDR3 shown in SEQ ID NO: 4;
(2) CDR1 shown in SEQ ID NO: 6, CDR2 shown in SEQ ID NO: 7, and CDR3 shown in SEQ ID NO: 8;
(3) CDR1 shown in SEQ ID NO: 10, CDR2 shown in SEQ ID NO: 11, and CDR3 shown in SEQ ID NO: 12;
(4) CDR1 shown in SEQ ID NO: 14, CDR2 shown in SEQ ID NO: 15, and CDR3 shown in SEQ ID NO: 16;
(5) CDR 1 shown in SEQ ID NO: 2, CDR2 shown in SEQ ID NO: 18, and CDR3 shown in SEQ ID NO: 19;
(6) CDR 1 shown in SEQ ID NO: 21, CDR2 shown in SEQ ID NO: 22, and CDR3 shown in SEQ ID NO: 23;
(7) CDR1 shown in SEQ ID NO: 25, CDR2 shown in SEQ ID NO: 26, and CDR3 shown in SEQ ID NO: 27;
(8) CDR1 shown in SEQ ID NO: 29, CDR2 shown in SEQ ID NO: 30, and CDR3 shown in SEQ ID NO: 31;
(9) CDR1 shown in SEQ ID NO: 33, CDR2 shown in SEQ ID NO: 34, and CDR3 shown in SEQ ID NO: 35;
(10) CDR 1 shown in SEQ ID NO: 37, CDR2 shown in SEQ ID NO: 30, and CDR3 shown in SEQ ID NO: 38; and
(11) CDR 1 shown in SEQ ID NO: 40, CDR2 shown in SEQ ID NO: 41, and CDR3 shown in SEQ ID NO: 42;
and any one of the above amino acid sequences further comprises a derivative sequence that is optionally added, deleted, modified and/or substituted with at least one (such as 1-3, preferably 1-2, more preferably 1) amino acid and may retain the ability to bind to PD-L1.

2. (canceled)

3. The antibody-drug conjugate or a pharmaceutically acceptable salt thereof according to claim 1, wherein the TLR agonist is a TLR7 agonist.

4. The antibody-drug conjugate or a pharmaceutically acceptable salt thereof according to claim 3, wherein the TLR7 agonist comprises: SZU-101:

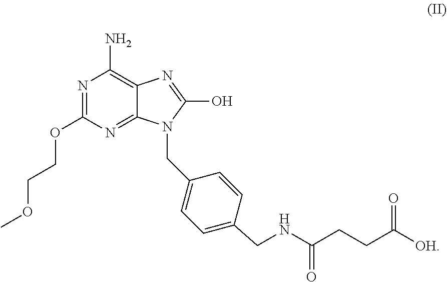 [SMILES: COCCOc1nc(N)c2nc(O)n(Cc3ccc(CNC(=O)CCC(=O)O)cc3)c2n1]

5. The antibody-drug conjugate or a pharmaceutically acceptable salt thereof according to claim 1, wherein the antibody-drug conjugate or a pharmaceutically acceptable salt thereof is used to prepare a composition or preparation, and the composition or preparation is used for:
(a) promoting the maturation of dendritic cells;
(b) enhancing the function of tumor-infiltrating cytotoxic cells (CD8+ T cells and NK cells);
(c) promoting the expression of granzyme B and IFN-γ in tumor-infiltrating cytotoxic cells;
(d) promoting the repolarization of tumor-associated macrophages;
(e) reducing infiltration of TGF-β+ macrophages;
(f) promoting the infiltration of IFN-γ+ CD 4+ T cells;
(g) promoting the expression of PD-L1 by intratumoral macrophages;
(h) targeting and reconstituting the tumor immune microenvironment;
(i) increasing the level of PD-L1 in tumor cells; and/or
(j) treating a tumor with a moderate or low PD-L1 expression.

6. A PD-L1 nanobody, wherein the PD-L1 nanobody specifically binds to human PD-L1, and the complementarity determining region CDR of the VHH chain in the nanobody is selected from the group consisting of:
(1) CDR1 shown in SEQ ID NO: 2, CDR2 shown in SEQ ID NO: 3, and CDR3 shown in SEQ ID NO: 4;
(2) CDR1 shown in SEQ ID NO: 6, CDR2 shown in SEQ ID NO: 7, and CDR3 shown in SEQ ID NO: 8;
(3) CDR1 shown in SEQ ID NO: 10, CDR2 shown in SEQ ID NO: 11, and CDR3 shown in SEQ ID NO: 12;
(4) CDR1 shown in SEQ ID NO: 14, CDR2 shown in SEQ ID NO: 15, and CDR3 shown in SEQ ID NO: 16;
(5) CDR1 shown in SEQ ID NO: 2, CDR2 shown in SEQ ID NO: 18, and CDR3 shown in SEQ ID NO: 19;
(6) CDR1 shown in SEQ ID NO: 21, CDR2 shown in SEQ ID NO: 22, and CDR3 shown in SEQ ID NO: 23;
(7) CDR1 shown in SEQ ID NO: 25, CDR2 shown in SEQ ID NO: 26, and CDR3 shown in SEQ ID NO: 27;
(8) CDR1 shown in SEQ ID NO: 29, CDR2 shown in SEQ ID NO: 30, and CDR3 shown in SEQ ID NO: 31;
(9) CDR1 shown in SEQ ID NO: 33, CDR2 shown in SEQ ID NO: 34, and CDR3 shown in SEQ ID NO: 35;
(10) CDR1 shown in SEQ ID NO: 37, CDR2 shown in SEQ ID NO: 30, and CDR3 shown in SEQ ID NO: 38; and
(11) CDR 1 shown in SEQ ID NO: 40, CDR2 shown in SEQ ID NO: 41, and CDR3 shown in SEQ ID NO: 42;
and any one of the above amino acid sequences further comprises a derivative sequence that is optionally added, deleted, modified and/or substituted with at least one (such as 1-3, preferably 1-2, more preferably 1) amino acid and may retain the ability to bind to PD-L1.

7. The PD-L1 nanobody according to claim 6, wherein the nanobody specifically binding to human PD-L1 is a humanized nanobody specifically binding to human PD-L1, and the nanobody comprises a VHH chain, the amino acid sequence of which is selected from the group consisting of:
(a) an amino acid sequence as shown in SEQ ID NOs: 43, 44, 45, 46, 47, 48, 49, 50, 51, 52, 53, 54, 55, 56, 57, 58, 59, 60, 61, 62, 63, 64, 65, 66, 67, 68, 69, 70, 71;
(b) a derivative antibody or an active fragment formed by adding one or more amino acids, substituting one or more amino acids, or deleting 1-3 amino acids in the amino acid sequences of (a), wherein the derivative antibody or active fragment retains the ability to specifically bind to PD-L1.

8. The PD-L1 nanobody according to claim 6, wherein the nanobody that specifically binds to human PD-L1 comprises a VHH chain, the amino acid sequence of which is selected from the group consisting of:
(a) an amino acid sequence as shown in SEQ ID NO: 72, 73, 74, 75, 76, 77, 78, 79, 80, 81, 82, 83, 84, 85, 86, 87, 88, 89, 90, 91, 92, 93, 94, 95, 96 or 97;
(b) a derivative antibody or an active fragment formed by adding one or more amino acids, substituting one or more amino acids, or deleting 1-3 amino acids in the amino acid sequence of (a), wherein the derivative antibody or active fragment retains the ability to specifically bind to PD-L1.

9. A pharmaceutical composition, wherein the pharmaceutical composition comprises:
(a) the PD-L1 nanobody according to claim 6, or an antibody-drug conjugate comprising the PD-L1 nanobody or a pharmaceutically acceptable salt thereof; and
(b) a pharmaceutically acceptable carrier.

10. The pharmaceutical composition according to claim 9, wherein the pharmaceutical composition is used to treat a tumor with a low expression of PD-L1.

11. An immunoconjugate, wherein the immunoconjugate comprises:
(a) the antibody-drug conjugate according to claim 1; and
(b) another coupling moiety.

12. A fusion protein, wherein the fusion protein comprises:
(a) the PD-L1 nanobody according to claim 6; and
(b) optionally a polypeptide molecule and protein fragment having therapeutic function.

13. A multispecific antibody, wherein the multispecific antibody comprises:
(a) the PD-L1 nanobody according to claim 6; and
(b) optionally an antibody molecule targeting a second antigen.

14. A medical kit, wherein the medical kit comprises:
(1) a first container, in which the PD-L1 nanobody according to claim 6, and a pharmaceutically acceptable carrier are contained;
(2) a second container, in which a TLR7 agonist, and a pharmaceutically acceptable carrier are contained;
and (3) optionally an instruction manual.

15. (canceled)

16. A method for preventing or treating a tumor, wherein the method comprises administering to a subject in need the PD-L1 nanobody of claim 6, a composition thereof, an immunoconjugate thereof, a fusion protein thereof, or an antibody-drug conjugate thereof, or a combination thereof.

17. The method of claim 16, wherein the tumor is a tumor expressing PD-L1.
